(19) United States
(12) Patent Application Publication
(10) Pub. No.: US 2007/0123537 A1
(43) Pub. Date: May 31, 2007

(54) Quinazoline derivatives for the treatment of herpesviral infections

(75) Inventor: Thomas Herget, Darmstadt (DE)

Correspondence Address: BROOKS KUSHMAN P.C., 1000 TOWN CENTER, TWENTY-SECOND FLOOR, SOUTHFIELD, MI, 48075, US

(73) Assignee: GPC Biotech AG, Munich (DE)

(21) Appl. No.: 10/595,307

(22) PCT Filed: Oct. 5, 2004
(86) PCT No.: PCT/EP04/11118

§ 371 Date: Dec. 16, 2006

(30) Foreign Application Priority Data
Oct. 6, 2003 (EP) 03022647.6

(51) Int. Cl. A61K 31/517; C07D 405/14; C07D 403/02; C07D 239/94

(52) U.S. Cl. 514/252.17; 514/266.24; 514/266.4; 544/284; 544/293

(57) ABSTRACT

The present invention relates to phenyl-quinazolinyl-amine derivatives and pharmaceutically acceptable salts thereof and pharmaceutical compositions comprising at least one of these derivatives and/or pharmaceutically salts thereof, as well as the use of these derivatives for the prophylaxis and/or treatment of herpesviral induced infections, including opportunistic infections, especially for the prophylaxis and/or treatment of infections and diseases induced by HCMV.

The present invention relates to quinazoline derivatives and pharmaceutically acceptable salts thereof and pharmaceutical compositions comprising at least one of these derivatives and/or pharmaceutically salts thereof, as well as the use of these derivatives for the prophylaxis and/or treatment of herpesviral induced infections, including opportunistic infections.

### BACKGROUND OF THE INVENTION

Human Cytomegalovirus (HCMV) is a highly specific β-herpesvirus. Primary infection of healthy children and adults is usually asymptomatic, with a minority of cases developing a mononucleose-like syndrome. In contrast, congenital infection (U.S. 0.2%-2.2% per live birth; aprox. 40,000 per year) leads to several neurological defects in 10-15% of infected neonates. Immunocompromised patients represent another host group facing serious disease complications caused by HCMV infection or reactivation of a persistent infection. Up to 40% of the AIDS patients, for example, develop retinitis, pneumonitis, gastroenteritis or disseminated HCMV disease. In addition, allograft recipients (20,000 allograft transplantations per year in the U.S.) are often infected (or superinfected) by virus from the transplanted organ.

Clinical symptoms in the posttransplant period include prolonged fever, leukopenia, thrombocytopenia, atypical lymphocytosis, elevated hepatic transaminases and decreased graft survival. In bone marrow transplantations, HCMV infection is associated with high mortality rates (80-90% for untreated HCMV pneumonia).

Current approaches to develop therapeutics against Cytomegalovirus (CMV) have focused on antiviral agents per se; for example viral polymerase inhibitors. In fact, high mortality rates have been dramatically reduced by new antiviral agents. Current CMV therapeutics possess severe drawbacks, however. For example, Fomivirsen (Vitravene®, formerly ISIS 2922) is typically administered by injection directly into the eye every 2 or 4 weeks. Ganciclovir is available for intravenous (Cytovene®) or oral administration, and as an implant in the case of retinitis; unfortunately, toxic complications including leukopenia and thrombocytopenia frequently develop. Foscamet (Foscavir®; phosphonoformic acid), another antiviral agent, exhibits considerable renal toxicities and is only available in intravenous form (which is also true for Cidofovir (Vistide®), another CMV therapeutic). In addition, CMV replication resumes soon after Ganciclovir and Foscarnet treatment is halted. Finally, Ganciclovir- and Foscarnet-resistant strains of CMV are emerging.

Although treatment of HCMV-induced disease has been improved with these inhibitors of the viral polymerase and preemptive or early antiviral therapy in transplant patients, there is a urgent need in the art for a new class of HCMV therapeutics with better oral bioavailability and reduced toxic effects. This is especially true in the treatment of retinitis in AIDS patients, where CMV infection must be controlled for long periods of time. Furthermore, there is a need for HCMV therapeutics, which could be used for treating drug resistant strains of CMV, especially of Ganciclovir- and/or Foscamet-resistant strains.

Thus, it is object of the present invention to provide compounds and/or pharmaceutically acceptable salts thereof which can be used as pharmaceutically active agents, especially for the prophylaxis and/or treatment of herpesviral infections and/or associated diseases, including opportunistic infections, a method to treat herpesviral induced infections by means of those compounds, as well as compositions comprising at least one of those compounds and/or pharmaceutically acceptable salts thereof as pharmaceutically active ingredients.

The object of the present invention is solved by the teaching of the independent claims. Further advantageous features, aspects and details of the invention are evident from the dependent claims, the description, and the examples of the present application.

One aspect of the present invention is related to novel quinazoline compounds of the general formula (I): 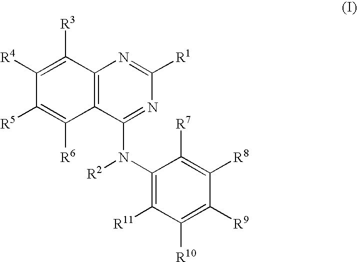
wherein:
R¹ is selected from the group consisting of:
   —H, C₁-C₆-alkyl, aryl, or 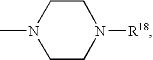
   wherein R¹⁸ is selected from the group consisting of 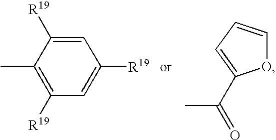
   wherein R¹⁹ is independently selected from the group consisting of —H, —F, —Cl, —Br, —I, —NO₂, —NH₂ or —CF₃,
R² is selected from the group consisting of:
   —H and C₁-C₆-alkyl,
R³, R⁴, R⁵ and R⁶ are independently selected from the group consisting of:
   —H, C₁-C₆-alkyl, C₁-C₆-alkoxy, phenoxy, —F, —Cl, —Br, —I, —OH, —CN, —NR¹²R^{12′}, —N═N—R¹³, —NH—C(O)—R¹⁴, —NO₂, —C≡C—R¹⁵, —C(R²⁰)₃, or —CH(R²⁰)₂ or 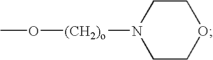
   wherein o is selected to an integer from 0 to 6, and wherein
      R¹² and R^{12′}are independently selected from the group consisting of:
         —H and C₁-C₆-alkyl,
      R¹³ and R¹⁴ are independently selected from the group consisting of
         —H, C₁-C₆-alkyl,
      —(CH₂)ₙ—R¹⁶, wherein n is selected to be an integer from 1 to 6 and R¹⁶ is selected from the group consisting of:
         —OH, —NH₂, or —CN,
      —(CH₂)ₘ—CH═CH₂, wherein m is selected to be an integer from 0 to 6, 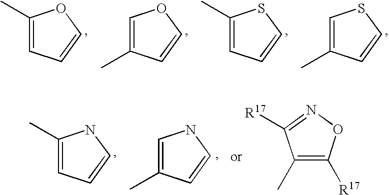
         wherein R¹⁷ is selected from the group consisting of —H, C₁-C₆-alkyl,
      C₃-C₆-cycloalkyl,
      phenyl substituted cyclopropyl, wherein the phenyl group is optionally substituted by one or two substituents R¹⁸, and R¹⁸ is independently selected from the group consisting of:
         —F, —Cl, —Br, —I, —CN, —OH, —NH₂
   R¹⁵ is selected from the group consisting of:
      —H and C₁-C₆-alkyl,
   or wherein R⁴ and R⁵ together form one of the ring systems represented by the formulas 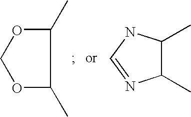
   and
   R²⁰ is independently selected from —F, —Cl, and —Br,

R⁷, R⁸, R⁹, R¹⁰, and R¹¹ are independently selected from the group consisting of:
H, C₁-C₆-alkyl, C₁-C₆-alkoxy,—F, —Cl, —Br, —I , —OH, —CN, —NR¹²R^{12′}, —N═N—R¹³, —NH—C(O)—R¹⁴, —NO₂, —C≡C—R¹⁵, —C(R²⁰)₃, or —CH(R²⁰)₂, 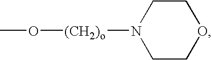
   wherein o is selected to an integer from 0 to 6, 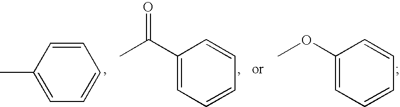
   wherein
   R¹², R^{12′}, R¹³, R¹⁴, R¹⁵ and R²⁰ represent groups as defined for R³ to R⁶,
      or wherein

R⁸ and R⁹ together form a together form a ring system represented by the formulas 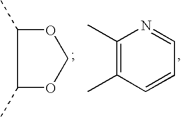
and/or pharmaceutically acceptable salts of the above compounds.

As used in the present invention, the term C₁-C₆ alkyl is meant to include the following linear or branched alkyls:
methyl, ethyl, n-propyl, iso-propyl, n-butyl, sec-butyl, iso-butyl, tert.-butyl, —C₅H₁₁, —CH₂—C(CH₃)₃, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₂H₄—CH(CH₃)₂, —C₆H₁₃, —C₃H₆—CH(CH₃)₂, —C₂H₄—CH(CH₃)—C₂H₅, —CH(CH₃)C₄H₉, —CH₂—CH(CH₃) —C₃H₇, —CH(CH₃)—CH₂—CH(CH₃)₂, —CH(CH₃)—CH(CH₃)—C₂H₅, —CH₂—CH(CH₃—CH(CH₃)₂, —CH₂—C(CH₃)₂—C₂H₅, —C(CH₃)₂—C₃H₇, —C(CH₃)₂, —CH(CH₃)₂, —C₂H₄—C(CH₃)₃ or —CH(CH₃)—C(CH₃)₃.

The term C₁-C₄-alkyl is therefore meant to include the respective subgroup out of the above groups.

Similarly, the term C₁-C₆ alkoxy is meant to include the following linear or branched alkoxy-groups:
methoxy, ethoxy, n-propoxy, iso-propoxyl, n-butoxy, sec-butoxy, iso-butoxy, tert.-butoxy, —O—C₅H₁₁, —O—CH₂—C(CH₃)₃, —O—CH(CH₃)—C₃H₇, —O—CH₂—CH(CH₃)—C₂H₅, —O—CH(CH₃)—CH(CH₃)₂, —O—C(CH₃)₂—C₂H₅, —O—CH₂—C(CH₃)₃, —O—C₂H₄—CH(CH₃)₂, —O—C₆H₁₃, —O—C₃H₆—CH(CH₃)₂, —O—C₂H₄—CH(CH₃—C₂H₅, —O—CH(CH₃)—C₄H₉, —O—CH₂—CH(CH₃)—C₃H₇, —O—CH(CH₃) —CH₂—CH(CH₃)₂, —O—CH(CH₃)—CH(CH₃)—C₂H₅, —O—CH₂—CH(CH₃)—CH(CH₃)₂, —O—CH₂—C(CH₃)₂—C₂H₅, —O—C(CH₃)₂—C₃H₇, —O—C(CH₃)₂—CH(CH₃)₂, —O—C₂H₄—C(CH₃)₃ or —O—CH(CH₃)—C(CH₃)₃.

The term aryl is meant to include phenyl, benzyl or naphtyl. Similarly, the C₃-C₆-cycloalkyl is meant to include cyclolalkanes like cyclopropyl, cyclobutyl, cyclopentyl or cyclohexyl.

In a preferred embodiment of the present invention R¹ in the compounds according to general formula (I) is selected from the group consisting of —H or C₁-C₄-alkyl, preferably R¹ is —H or methyl.

In a further preferred embodiment of the present invention R² in the compounds according to general formula (I) is selected from the group consisting of —H or C₁-C₄-alkyl, and preferably is —H.

In a further preferred embodiment of the present invention R³, R⁴, R⁵ and R⁶ in the compounds according to general formula (I) are independently selected from the group consisting of:
—H, C₁-C₆-alkyl, C₁-C₆-alkoxy, phenoxy, —F, —Cl, —Br, —I, —OH, —CN, —NR ¹²R^{12′}, —NH—C(O)—R¹⁴, —NO₂, —CF₃, or 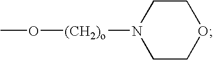
   wherein o is selected to an integer from 0 to 4, preferably from 2 to 4, most preferably is 3,
   and wherein
   R¹² and R^{12′}are independently selected from the group consisting of —H or methyl,
   R¹⁴ is selected from the group consisting of
C₁-C₆-alkyl,
   —(CH₂)ₘ—CH≡CH₂, wherein m is selected to be an integer from 0 to 2 and preferably is 0,
   cyclopropyl, cyclobutyl, cyclopentyl, and cyclohexyl, and phenyl substituted cyclopropyl,
or wherein R⁴ and R⁵ together form a ring system represented by the formula

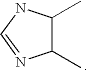

In a further preferred embodiment of the present invention, R⁷, R⁸, R⁹, R¹⁰ , and R¹¹ in the compounds according to general formula (I) are independently selected from the group consisting of:
—H, C₁-C₆-alkyl, C₁-C₆-alkoxy, —F, —Cl, —Br, —I, —OH, —N═N—R¹³, —NH—C(O)—R¹⁴, —NO₂, —C≡C—H, 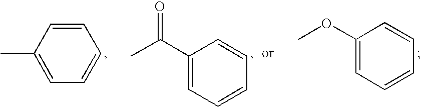
wherein
   R¹³ and R¹⁴ are independently selected from the group consisting of
      C₁-C₆-alkyl, and preferably are methyl or ethyl,
      —(CH₂)ₙ,—R¹⁶, wherein n is selected to be an integer from 1 to 6 and R¹⁶ is selected from the group consisting of:
         —NH₂ and —CN,
      —CH═CH₂, 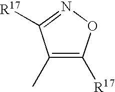
         wherein R¹⁷ is selected from the group consisting of: —H and methyl,
      cyclopropyl, cyclobutyl, cyclopentyl, cyclohexyl, phenyl substituted cyclopropyl,
   or wherein R⁸ and R⁹ together form one of the ring systems represented by the formulas

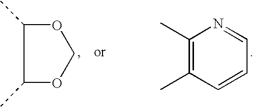

In a further preferred embodiment of the present invention, R³ and R⁶ in the compounds according to general formula (I) represent —H and R⁴ and R⁵ are independently selected from the group consisting of:
—H, C₁-C₄-alkyl, and preferably are —H or methyl.

In a further preferred embodiment of the present invention, R⁷, R⁸, R⁹, R¹⁰, and R¹¹ in the compounds according to general formula (I) are independently selected from the group consisting of: —H, C₁-C₆-alkyl, C₁-C₆alkoxy, —F, —Cl, —Br, —I, —OH, —CN, and —NR¹²R^{12′}, wherein R¹² and R¹²′ are independently selected from —H or methyl and preferably are both —H.

In a further preferred embodiment of the present invention, R⁷, R⁸, R⁹, R¹⁰, and R¹¹ in the compounds according to general formula (I) are independently selected from the group consisting of: —H, —F, —Cl, —Br, and —I.

In yet another preferred embodiment of the present invention at least two of the groups R⁷, R⁸, R⁹, R¹⁰, and R¹¹ in the compounds according to general formula (I) are —H, preferably two or three of the groups R⁷, R⁸, R⁹, R¹⁰, and R¹¹ are —H.

In yet another preferred embodiment of the present invention four of the groups R⁷, R⁸, R⁹, R¹⁰, and R¹¹ in the compounds according to general formula (I) are —H.

In yet another preferred embodiment of the present invention R⁷, R⁹, R¹⁰, and R¹¹ in the compounds according to general formula (I) are —H, or wherein R⁷, R¹⁰ and R¹¹ are —H, or wherein R⁸ and R¹⁰ are —H.

In yet another preferred embodiment of the present invention, those groups out of the group R⁷, R⁸, R⁹, R¹⁰, and R¹¹ in the compounds according to general formula (I) which are not —H are selected from the group consisting of —F, —Cl, —Br and —I.

In those embodiments, in which only one of the residues R⁷, R⁸, R⁹, R¹⁰, and R¹¹ in the compounds according to general formula (I) is a group other than —H, this group can be bonded in ortho, para or meta position to the phenyl group. If two residues out of R⁷, R⁸, R⁹, R¹⁰, and R¹¹ in the compounds according to general formula (I) are selected to be other than —H, these two residues can be bonded to the phenyl group in 2 and 3, 2 and 4, 2 and 5, 2 and 6, 3 and 4, or 3 and 5 position. If three of the groups R⁷, R⁸, R⁹, R¹⁰, and R¹¹ are selected to be other than —H, these groups can be bonded to the phenyl group in 2, 3 and 4 position, 2, 4 and 5 position, or 2, 4 and 6 position. If four residues out of the group of R⁷, R⁸, R⁹, R¹⁰, and R¹¹ are selected to other than —H, these residues can be bonded to the phenyl group in 2, 3, 4, and 5 position or 2, 3, 5 and 6 position.

In yet another preferred embodiment of the present invention, compounds according to formula (I) are selected from the group comprising:
Compound 1: (3-Nitro-phenyl)-quinazolin-4-yl-amine,
Compound 2: (3-Bromo-phenyl)-quinazolin-4-yl-amine,
Compound 3: (6,7-Dimethoxy-quinazolin-4-yl)-[(3-(3,5-dimethyl-isoxyzol-4-ylazo)-phenyl]-amine,
Compound 4: Furan-2-carboxylic acid [4-(6,7-dimethoxy-quinazolin-4-yl-amino)-phenyl]-amide,
Compound 5: Furan-2-carboxylic acid [3-(6,7-dimethoxy-quinazolin-4-yl-amino)-phenyl]-amide,
Compound 6: 2-Cyano-N-[4-(6,7-dimethoxy-quinazolin-4-yl-amino)-phenyl]acetamide,
Compound 7: 2-Cyano-N-[3-(6,7-dimethoxy-quinazolin-4-yl-amino)-phenyl]acetamide,
Compound 8: (3-Bromo-phenyl)-(6-methoxy-quinazolin-4-yl)-amine,
Compound 9: (3-Bromo-phenyl)-(6-methyl-quinazolin-4-yl)-amine,
Compound 10: 6-Amino-hexanoic acid [4-(6,7-dimethoxy-quinazolin-4-yl)-phenyl]-amide,
Compound 11: 6-Amino-hexanoic acid [3-(6,7-dimethoxy-quinazolin-4-yl)-phenyl]-amide,
Compound 12: (3-Bromo-phenyl)-(6,7-dimethoxy-quinazolin-4-yl)-amine,
Compound 13: (6,7-Dimethoxy-2-methyl-quinazolin-4-yl)-(3-nitro-phenyl)-amine,
Compound 14: (6,7-Dimethoxy-2-methyl-quinazolin-4-yl)-(4-nitro-phenyl)-amine,
Compound 15: N-[4-(3-Bromo-phenylamino)-quinazolin-6-yl]-propionamide,
Compound 16: (3-Bromo-phenyl)-(6,7-diethoxy-quinazolin-4-yl)-amine,
Compound 17: (6,7-diethoxy-quinazolin-4-yl)-(3-hydroxy-phenyl)-amine,
Compound 18: N-[4-(3-Bromo-phenylamino)-quinazolin-6-yl]-propargylamide,
Compound 19: (3-Choro-phenyl)-(6,7-dimethoxy-quinazolin-4-yl)-amine,
Compound 20: N⁴-(3-Bromo-phenyl)-N⁶-methyl-quinazoline-4,6,7-triamine,
Compound 21: (3-Bromo-4-methoxy-phenyl)-(6,7-dimethoxy-quinazolin-4-yl)-amine,
Compound 22: N⁴-(3-Bromo-phenyl)-quinazoline-4,6,7-triamine,
Compound 23: (3,5-Bis-trifluormethyl-phenyl-quinazolin-4-yl)-amine,
Compound 24: (4-Fluoro-phenyl)-(6-methyl-quinazolin)-yl)-amine,
Compound 25: (3-Chloro-phenyl)-(6-methyl-quinazolin-4-yl)-amine,
Compound 26: (6-Methyl-quinazolin-4-yl)-phenyl-amine,
Compound 27: (3,4-Dimethyl-phenyl)-(6-methyl-quinazolin-4-yl)-amine,
Compound 28: (2-Hydroxy-4-methyl-phenyl)-(6-methyl-quinazolin-4-yl)-amine,
Compound 29: (6,7-Dimethoxy-quinazolin-4-yl)-(4-fluoro-phenyl)-amine,
Compound 30: (3,4-Dimethyl-phenyl)-(6,7-dimethoxy-quinazolin-4-yl)-amine,
Compound 31: (4-Bromo-phenyl)-(6,7-dimethoxy-quinazolin-4-yl)-amine,
Compound 32: Benzo[1,3]-dioxol-5-yl-(6,7-dimethoxy-quinazolin-4-yl)-amine,
Compound 33: (3-Bromo-phenyl)-(6,7-dimethoxy-2-methyl-quinazolin-4-yl)-amine,
Compound 34: (3-Chloro-5-hydroxy-phenyl)-(6-methyl-quinazolin-4-yl)-amine,
Compound 35: N-[4-(Quinazolin-4-yl-amino)-phenyl]-acetamide,
Compound 36: Phenyl-[4-(quinazolin-4-yl-amino)-phenyl]-methanone,
Compound 37: (3,4-Dichloro-phenyl)-quinazolin-4-yl-amine,
Compound 38: (4-Chloro-2-hydroxy-phenyl)-quinazolin-4-yl-amine,
Compound 39: (2-Hydroxy-4-methyl-phenyl)-quinazolin-4-yl-amine,
Compound 40: (3,4-Dimethyl-phenyl)-quinazolin-4-yl-amine,
Compound 41: Benzo[1,3]-dioxol-5-yl-(6-methyl-quinazolin-4-yl)-amine,
Compound 42: (3-Chloro-4-methyl-phenyl)-(6-methyl-quinazolin-4-yl)-amine,
Compound 43: (4-Bromo-phenyl)-(6-methyl-quinazolin-4-yl)-amine,
Compound 44: N-[4-(6-Methyl-quinazolin-4-yl-amino)-phenyl]-acetamide,
Compound 45: (4-lodo-phenyl)-(6-methyl-quinazolin-4-yl)-amine,
Compound 46: Phenyl-[4-(6-methyl-quinazolin-4-yl-amino)-phenyl]-methanone,
Compound 47: (4-Phenoxy-phenyl)-quinazolin-4-yl-amine,
Compound 48: (3-Hydroxy-phenyl)-(6-methyl-quinazolin-4-yl)-amine,
Compound 49: (3,4-Dichloro-phenyl)-(6-methyl-quinazolin-4-yl)-amine,
Compound 50: N-[4-(8-Methyl-quinazolin-4-yl-amino)-phenyl]-acetamide,
Compound 51: (4-lodo-phenyl)-(8-methyl-quinazolin-4-yl)-amine,
Compound 52: (4-Phenoxy-phenyl)-(8-methyl-quinazolin-4-yl)-amine,
Compound 53: (3,4-Dichloro-phenyl-(8-methyl-quinazolin-4-yl)-amine,
Compound 54: Benzo[1,3]-dioxol-5-yl-(8-methyl-quinazolin-4-yl)-amine,
Compound 55: (3-Chloro-4-methyl-phenyl)-(8-methyl-quinazolin-4-yl)-amine,
Compound 56: (4-Bromo-phenyl)-(8-methyl-quinazolin-4-yl)-amine,
Compound 57: (3,4-Dimethoxy-phenyl)-(8-methyl-quinazolin-4yl)-amine,
Compound 58: (3-Chloro-6-hydroxy-phenyl)-(8-methyl-quinazolin-4-yl)-amine,
Compound 59: (2-Methoxy-methyl-phenyl)-(8-methyl-quinazolin-4-yl)-amine,
Compound 60: (4-Chloro-phenyl)-(8-methyl-quinazolin-4-yl)-amine,
Compound 61: (8-Methyl-quinazolin-4-yl)-phenyl-amine
Compound 62: (3-Chloro-phenyl-(8-methyl-quinazolin-4-yl)-amine,
Compound 63: (4-Fluoro-phenyl)-(8-methyl-quinazolin-4-yl)-amine,
Compound 64: (3,5-Bis-fluoromethyl-phenyl)-(8-methyl-quinazolin-4-yl)-amine,
Compound 65: (3-Bromo-phenyl)-(8-methyl-quinazolin-4-yl)-amine,
Compound 66: (3,5-Bis-fluoromethyl-phenyl)-(6-methyl-quinazolin-4-yl)-amine,
Compound 67: (4-Chloro-phenyl)-(6-methyl-quinazolin-4-yl)-amine,
Compound 68: Phenyl-[4-(8-methyl-quinazolin-4-yl-amino)-phenyl]-methanone,
Compound 69: (3-Bromo-phenyl)-(7-fluoro-quinazolin-4-yl)-amine,
Compound 70: (3-Chloro-phenyl)-(7-fluoro-quinazolin-4-yl)-amine,
Compound 71: (3-Ethyl-phenyl)-quinazolin-4-yl-amine,
Compound 72: (4-lodo-phenyl)-quinazolin-4-yl-amine,
Compound 73: (4-Phenoxy-phenyl)-quinazolin-4-yl-amine,
Compound 74: (4-Fluoro-phenyl)-(7-fluoro-quinazolin-4-yl)-amine,
Compound 75: (3-Fluoro-phenyl)-quinazolin-4-yl-amine,
Compound 76: (2,4-Dichloro-phenyl)-quinazolin-4-yl-amine,
Compound 77: (3-Hydroxy-phenyl)-quinazolin-4-yl-amine,
Compound 78: (3-Fluoro-4-methyl-phenyl)-(6-methyl-quinazolin-4-yl)-amine,
Compound 79: (3-Fluoro-phenyl)-(6-methyl-quinazolin-4-yl)-amine,
Compound 80: (3-Chloro-6-methyl-phenyl)-(7-fluoro-quinazolin-4-yl)-amine,
Compound 81: Biphenyl-4-yl-quinazolin-4-yl-amine,
Compound 82: (3,4-Dimethoxy-phenyl)-quinazolin-4-yl-amine,
Compound 83: (6-Fluoro-quinazolin-4-yl)-(2-methyl-phenyl)-amine,
Compound 84: Phenyl-quinazolin-4-yl-amine,
Compound 85: (3-Chloro-6-methyl-phenyl)-quinazolin-4-yl-amine,
Compound 86: (4-Ethyl-phenyl)-quinazolin-4-yl)-amine,
Compound 87: (3-Chloro-phenyl)-quinazolin-4-yl-amine,
Compound 88: (4-Fluoro-phenyl)-quinazolin-4-yl-amine,
Compound 89: (3-Ethyl-phenyl)-(6-methyl-quinazolin-4-yl)-amine,
Compound 90: (7-Fluoro-quinazolin-4-yl)-phenyl-amine,
Compound 91: (3-Chloro-2-methyl-phenyl)-(6-methyl-quinazolinyl)-amine,
Compound 92: (4-Butyl-phenyl)-(6-methyl-quinazolin-4-yl)-amine,
Compound 93: (4-Hydroxy-6-methyl-phenyl)-quinazolin-4-yl-amine,
Compound 94: (3-Nitro-6-methyl-phenyl)-quinazolin-4-yl-amine,
Compound 95: (3-Chloro-2-methyl-phenyl)-quinazolin-4-yl-amine,
Compound 96: (4-Ethyl-phenyl)-(6-methyl-quinazolin-4-yl)-amine,
Compound 97: (4-Chloro-6-methyl-phenyl)-quinazolin-4-yl-amine,
Compound 98: (4-Bromo-phenyl)-quinazolin-4-yl-amine,
Compound 99: (3-Chloro-6-methyl-phenyl)-(8-methyl-quinazolin-4-yl)-amine,
Compound 100: (3-Chloro-6-methyl-phenyl)-(6-methyl-quinazolin-4-yl)-amine,
Compound 101: (3,4-Dimethoxy-phenyl)-(6-methyl-quinazolin-4-yl)-amine,
Compound 102: (3,4-Dimethoxy-phenyl)-(6,7-dimethoxy-quinazolin-4-yl)-amine,
Compound 103: (6-Methyl-quinazolin-4-yl)-(4-trifluoromethyl-phenyl)-amine,
Compound 104: (2-Fluoro-phenyl)-(7-fluoro-quinazolin-4-yl)-amine,
Compound 105: (3-Chloro-phenyl)-(6,7-dimethoxy-2-methyl-quinazolin-4-yl)-amine,
Compound 106: (6,7-Dimethoxy-quinazolin-4-yl)-(3-trifluoromethyl-phenyl)-amine,
Compound 107: (3-Bromo-phenyl)-(6-nitro-quinazolin-4-yl)-amine,
Compound 108: Biphenyl-4-yl-(8-methyl-quinazolin-4-yl)-amine,
Compound 109: (8-Methyl-quinazolin-4-yl)-(4-trifluoromethyl-phenyl)-amine,
Compound 110: (2,4-Dichloro-phenyl)-(6-methyl-quinazolin-4-yl)-amine,
Compound 111: (4-Trifluoromethyl-phenyl)-quinazolin-4-yl-amine,
Compound 112: (2,3-Dimethyl-phenyl)-(6-methyl-quinazolin-4-yl)-amine,
Compound 113: (3,5-Bistrifluoromethyl-phenyl)-(7-fluoro-quinazolin-4-yl)-amine,)
Compound 114: (2,4-Dimethyl-phenyl)-(6-methyl-quinazolin-4-yl)-amine,
Compound 115: Benzo[1,3]-dioxol-5-yl-quinazolin-4-yl-amine,
Compound 116: (6,7-Dimethoxy-quinazolin-4-yl)-(4-nitro-phenyl)-amine,
Compound 117: (6-Methyl-quinazolin-4-yl)-(2,4,5-Trichloro-phenyl)-amine,
Compound 118: (3-Chloro-5-fluoro-phenyl)-(6,7-dimethoxy-quinazolin-4-yl)-amine,
Compound 119: (3-Bromo-phenyl)-(6-nitro-7-fluoro-quinazolin-4-yl)-amine,
Compound 120: N⁴-(3-Bromo-phenyl)-6-nitro-quinazoline-4,7-diamine,
Compound 121: N-[4-(3-Bromo-phenylamino)-7-cyclobutane-carbonylamino-quinazolin-6-yl]-cyclobutanecarboxylic acid amide,
Compound 122: N-[4-(3-Bromo-phenylamino)-7-cyclopentane-carbonylamino-quinazolin-6-yl]-cyclopentanecarboxylic acid amide,
Compound 123: N-[7-Acetylamino-5-(3-bromo-phenylamino)-quinazolin-6-yl]-acetamide,
Compound 124: 3-(6,7-dimethoxy-quinazolin-4-yl-amino-benzonitrile,
Compound 125: (3-Chloro-4-fluoro-phenyl)-[7-methoxy-6-(3-morpholin-4-yl-propoxy)-quinazolin-4-yl]-amine.

The inventive compounds according to general formula (I) and/or pharmaceutically acceptable salts thereof can be synthesized for example according to procedures described in EP0520722, WO 9633980, Tetrahedron Letters 1998, 39, 1785, Tetrahedron Letters 2000, 56, 9343 and references mentioned therein. The compounds (3-bromo-phenyl)-(6,7-diethoxy-quinazolin-4-yl)-amine and (3-bromo-phenyl)-(6,7-dimethoxy-quinazolin-4-yl)-amine are excluded from the scope of protection of this application by disclaimer.

Other aspects of the present invention relate to quinazoline derivatives according to the general formula (I) and pharmaceutically acceptable salts thereof as shown above for use as new pharmaceutically active agents, especially for the prophylaxis and/or treatment of herpesviral induced infections, including opportunistic infections , pharmaceutical compositions comprising at least one of these quinazoline derivatives as pharmaceutically active ingredient and a method for preventing and/or treating herpesviral induced infections, in mammals, including humans.

Surprisingly, it was found that quinazoline derivatives of the general formula (I) as well as pharmaceutically acceptable salts thereof, are effective against herpesviral infections and diseases at pharmaceutically acceptable concentrations. Furthermore, it was surprisingly found, that compounds of the general formula (I) as well as pharmaceutically acceptable salts thereof, are potent inhibitors for human and viral kinases, especially of human cellular protein kinases such as UL 97.

Additionally, the present invention relates to the use of the quinazoline derivatives of the general formula (I) as well as pharmaceutically acceptable salts thereof for the manufacturing of a pharmaceutical composition for the prophylaxis and/or treatment of herpesviral induced infections and diseases, including opportunistic diseases, especially infections and diseases induced by the human cytomegalovirus (HCMV).

The quinazoline derivatives as well as pharmaceutically acceptable salts thereof according to the present invention are effective against herpesviral induced infections. The herpesvirus family comprises the human herpesviruses 1 to 8 and different herpes viruses for various animal species as shown below in Table 1:

TABLE 1
Members of the herpesvirus family

| Subfamily | Genus | Human | Animal |
|---|---|---|---|
| α-herpesvirus | simplex virus | human herpesvirus 1 | bovine herpesvirus 2 |
| | | (herpes simplex virus 1) | |
| | varicella virus | human herpesvirus 2 | cercopithecine herpes virus |
| | | (herpes simplex virus 2) | 1, (herpes B virus) |
| | | | pseudorabiesvirus |
| | | human herpesvirus 3 | bovine herpesvirus 1 |
| | | (Varizelle Zoster virus) | equine-abortion virus |
| β-herpesvirus | cytomegalovirus | human herpesvirus 5 | |
| | | (HCMV) | |
| | muromegalovirus | | murine herpesvirus 1 |
| | Roseolovirus | human herpesvirus 6 | aotine herpesvirus 1, 3 |
| | | human herpesvirus 7 | |
| γ-herpesvirus | lymphocrytovirus | human herpesvirus 4 | cercopithecine herpesvirus 2 |
| | | (Epstein-Barr virus) | |
| | | | pongine herpesvirus 1 |
| | Rhadinovirus | human herpesvirus 8 | ateline herpesvirus 2 |
| | | | saimirine herpesvirus 1 |

Within the present invention herpesviruses may be selected from the group comprising: α-herpesviruses (Simplexvirus, Varicellavirus), β-herpesviruses (Cytomegalovirus also known as human herpesvirus 5, Muromegalovirus, Roseolovirus), or γ-herpesviruses (Lymphocryptovirus, Rhadinovirus). Examples for α-herpesviruses are Herpes simplex virus type 1 (human herpesvirus 1), Herpes simplex virus type 2 (human herpesvirus 2), Varicella Zoster virus (human herpesvirus 3). Examples for γ-herpesviruses are Epstein-Barr virus (human herpesvirus 4) or human herpesvirus type 8 (HHV8). More preferably, the herpesvirus is Herpes simplex virus type 1, or Varicella Zoster virus, or Epstein-Barr virus (EBV), or human cytomegalovirus (HCMV), or human herpesvirus 6, or human herpesvirus 7, or human herpesvirus type 8 (HHV8). Most preferably, the herpesvirus represents the α-herpesviruses Herpes simplex virus type 1, or Varicella Zoster virus, or the γ-herpesviruses Epstein-Barr virus, or Human Herpes virus type 8 or the β-herpesvirus human herpesvirus 5.

The present invention also provides a method for treating herpesviral induced infections, including opportunistic infections, in mammals, including humans, which method comprises administering to the mammal an amount of at least one of the quinazoline derivatives and/or pharmaceutically acceptable salts thereof effective to treat a herpesviral induced infection. Especially, the method is used for the prevention of treatment of infections and diseases induced by HCMV.

As used herein, a cellular kinase “inhibitor” refers to any compound capable of downregulating, decreasing, suppressing or otherwise regulating the amount and/or activity of a cellular kinase. Inhibition of these cellular kinases can be achieved by any of a variety of mechanisms known in the art, including, but not limited to binding directly to the cellular kinase polypeptide, denaturing or otherwise inactivating the cellular kinase, or inhibiting the expression of the gene (e.g., transcription to mRNA, translation to a nascent polypeptide, and/or final polypeptide modifications to a mature protein), which encodes the cellular kinase. Generally, cellular kinase inhibitors may be proteins, polypeptides, nucleic acids, small molecules, or other chemical moieties.

As used herein the term “inhibiting” or “inhibition” refers to the ability of an inhibitor to downregulate, decrease, reduce, suppress, inactivate, or inhibit at least partially the activity of an enzyme, or the expression of an enzyme and the virus replication. CMV therapeutics may be administered to cells from an individual in vitro, or may involve in vivo administration to the individual. Routes of administration of pharmaceutical preparations to an individual may include inhalation, oral and parenteral, including dermal, intradermal, intragastral, intracutan, intravasal, intravenous, intramuscular, intraperitoneal, intranasal, intravaginal, intrabuccal, percutan, rectal, subcutaneous, sublingual, topical or transdermal application, but are not limited the these ways of administration. For instance, the preferred preparations are in administratable form which is suitable for oral application. These administratable forms, for example, include pills, tablets, film tablets, coated tablets, capsules, powders and deposits. Administration to an individual may be in a single dose or in repeated administrations, and may be in any of a variety of physiologically acceptable salt forms, and/or with an acceptable pharmaceutical carrier, binder, lubricant, excipient, diluents and/or adjuvant. Pharmaceutically acceptable salt forms and standard pharmaceutical formulation techniques are well known to persons skilled in the art (see, for example, Remington's Pharmaceutical Sciences, Mack Publishing Co.).

As used herein, a “pharmaceutically effective amount” of a cellular kinase inhibitor is an amount effective to achieve the desired physiological result, either in cells treated in vitro or in a subject treated in vivo. Specifically, a pharmaceutically effective amount is an amount sufficient to inhibit, for some period of time, one or more of the clinically defined pathological processes associated with the viral infection. The effective amount may vary depending on the specific kinase inhibitor selected, and is also dependent on a variety of factors and conditions related to the subject to be treated and the severity of the infection. For example, if the inhibitor is to be administered in vivo, factors such as the age, weight and health of the patient as well as dose response curves and toxicity data obtained in preclinical animal work would be among those considered. If the inhibitor is to be contacted with the cells in vitro, one would also design a variety of pre-clinical in vitro studies to assess such parameters as uptake, half-life, dose, toxicity, etc. The determination of a pharmaceutically effective amount for a given agent is well within the ability of those skilled in the art.

As a result of the finding that UL97 plays an important role in the CMV infection process, a novel diagnostic assay useful for detecting the CMV infection of an individual (or cell) is identified. The diagnostic assay of the present invention involves providing a sample from an individual or providing cells, cell cultures and/or cell lysates, and detecting activity in the sample of said individual or in the cells, cell cultures or cell lysates of the said human cellular protein kinase Ul97. In one embodiment, deviations in the expression levels of the human cellular protein kinase UL97 in a test sample compared to known normal expression levels (e.g., determined from a sample from a healthy individual) will indicate presence of CMV.

It is apparent to a practitioner in the art that a sample useful for detecting CMV infection, whether of a subject individual or an isolated cell, refers to any cellular extract (including whole cells) from a tissue or body fluid (in the case of an individual) or cellular lysate (in the case of an isolated cell), which contains cellular components representative of cellular activity of one or more of the above-mentioned cellular kinases.

It is also apparent to a person of ordinary skill in the art that detection includes any method known in the art useful to indicate the presence, absence, or amount of a detection target. Such methods may include, but are not limited to, any molecular or cellular techniques, used singularly or in combination, including, but not limited to: hybridization and/or binding techniques, including blotting techniques and immunoassays; labeling techniques (chemiluminescent, colorimetric, fluorescent, radioisotopic); spectroscopic techniques; separations technology, including precipitations, electrophoresis, chromatography, centrifugation, ultrafiltration, cell sorting; and enzymatic manipulations (e.g., digestion).

The following compounds are preferred to be used within the methods or for the indications disclosed herein:
Compound 1: (3-Nitro-phenyl)-quinazolin-4-yl-amine,
Compound 2: (3-Bromo-phenyl)-quinazolin-4-yl-amine,
Compound 3: (6,7-Dimethoxy-quinazolin-4-yl)[(3-(3,5-dimethyl-isoxyzol4-ylazo)-phenyl]-amine,
Compound 4: Furan-2-carboxylic acid [4-(6,7-dimethoxy-quinazolin-4-yl-amino)phenyl]-amide,
Compound 5: Furan-2-carboxylic acid [3-(6,7-dimethoxy-quinazolin-4-yl-amino)phenyl]-amide,
Compound 6: 2-Cyano-N-[4-(6,7-dimethoxy-quinazolin-4-yl-amino)-phenyl]acetamide,
Compound 7: 2-Cyano-N-[3-(6,7-dimethoxy-quinazolin-4-yl-amino)-phenyl]acetamide,
Compound 8: (3-Bromo-phenyl)-(6-methoxy-quinazolin-4-yl)-amine,
Compound 9: (3-Bromo-phenyl)-(6-methyl-quinazolin-4-yl)-amine,
Compound 10: 6-Amino-hexanoic acid [4-(6,7-dimethoxy-quinazolin-4-yl)-phenyl]-amide,
Compound 11: 6-Amino-hexanoic acid [3-(6,7-dimethoxy-quinazolin-4-yl)-phenyl]-amide,
Compound 12: (3-Bromo-phenyl)-(6,7-dimethoxy-quinazolin-4-yl)-amine,
Compound 13: (6,7-Dimethoxy-2-methyl-quinazolin-4-yl)-(3-nitro-phenyl)-amine,
Compound 14: (6,7-Dimethoxy-2-methyl-quinazolin-4-yl)-(4-nitro-phenyl)-amine,
Compound 15: N-[4-(3-Bromo-phenylamino)-quinazolin-6-yl]-propionamide,
Compound 16: (3-Bromo-phenyl)-(6,7-diethoxy-quinazolin-4-yl)-amine,
Compound 17: (6,7-diethoxy-quinazolin-4-yl)-(3-hydroxy-phenyl)-amine,
Compound 18: N-[4-(3-Bromo-phenylamino)-quinazolin-6-yl]-propargylamide,
Compound 19: (3-Choro-phenyl)-(6,7-dimethoxy-quinazolin-4-yl)-amine,
Compound 20: N⁴-(3-Bromo-phenyl)-N⁶-methyl-quinazoline-4,6,7-triamine,
Compound 21: (3-Bromo-4-methoxy-phenyl)-(6,7-dimethoxy-quinazolin-4-yl)-amine,
Compound 22: N⁴-(3-Bromo-phenyl)quinazoline4,6,7-triamine,
Compound 23: (3,5-Bis-trifluormethyl-phenyl-quinazolin-4-yl)-amine,
Compound 24: (4-Fluoro-phenyl)-(6-methyl-quinazolin-4-yl)-amine,
Compound 25: (3-Chloro-phenyl)-(6-methyl-quinazolin-4-yl)-amine,
Compound 26: (6-Methyl-quinazolin-4-yl)-phenyl-amine,
Compound 27: (3,4-Dimethyl-phenyl)-(6-methyl-quinazolin-4-yl)-amine,
Compound 28: (2-Hydroxy-4-methyl-phenyl)-(6-methyl-quinazolin-4-yl)amine,
Compound 29: (6,7-Dimethoxy-quinazolin-4-yl)-(4-fluoro-phenyl)-amine,
Compound 30: (3,4-Dimethyl-phenyl)-(6,7-dimethoxy-quinazolin-4-yl)-amine,
Compound 31: (4-Bromo-phenyl)-(6,7-dimethoxy-quinazolin-4-yl)-amine,
Compound 32: Benzo[1,3]-dioxol-5-yl-(6,7-dimethoxy-quinazolin-4-yl)-amine,
Compound 33: (3-Bromo-phenyl)-(6,7-dimethoxy-2-methyl-quinazolin-4-yl)-amine,
Compound 34: (3-Chloro-5-hydroxy-phenyl)-(6-methyl-quinazolin-4-yl)-amine,
Compound 35: N-[4-(Quinazolin-4-yl-amino)-phenyl]-acetamide,
Compound 36: Phenyl-[4-(quinazolin-4-yl-amino)-phenyl]-methanone,
Compound 37: (3,4-Dichloro-phenyl)-quinazolin-4-yl-amine,
Compound 38: (4-Chloro-2-hydroxy-phenyl)-quinazolin-4-yl-amine,
Compound 39: (2-Hydroxy-4-methyl-phenyl)-quinazolin-4-yl-amine,
Compound 40: (3,4-Dimethyl-phenyl)quinazolin-4-yl-amine,
Compound 41: Benzo[1,3]-dioxol-5-yl-(6-methyl-quinazolin-4-yl)-amine,
Compound 42: (3-Chloro-4-methyl-phenyl)-(6-methyl-quinazolin-4-yl)-amine,
Compound 43: (4-Bromo-phenyl)-(6-methyl-quinazolin-4-yl)-amine,
Compound 44: N-[4-(6-Methyl-quinazolin-4-yl-amino)-phenyl]-acetamide,
Compound 45: (4-lodo-phenyl)-(6-methyl-quinazolin-4-yl)-amine,
Compound 46: Phenyl-[4-(6-methyl-quinazolin-4-yl-amino)-phenyl]-methanone,
Compound 47: (4-Phenoxy-phenyl)-quinazolin-4-yl-amine,
Compound 48: (3-Hydroxy-phenyl)-(6-methyl-quinazolin-4-yl)-amine,
Compound 49: (3,4-Dichloro-phenyl)-(6-methyl-quinazolin-4-yl)-amine,
Compound 50: N-[4-(8-Methyl-quinazolin-4-yl-amino)-phenyl]-acetamide,
Compound 51: (4-lodo-phenyl)-(8-methyl-quinazolin-4-yl)-amine,
Compound 52: (4-Phenoxy-phenyl)-(8-methyl-quinazolin-4-yl)-amine,
Compound 53: (3,4-Dichloro-phenyl)-(8-methyl-quinazolin-4-yl)-amine,
Compound 54: Benzo[1,3]-dioxol-5-yl-(8-methyl-quinazolin-4-yl)-amine,
Compound 55: (3-Chloro-4-methyl-phenyl)-(8-methyl-quinazolin-4-yl)-amine,
Compound 56: (4-Bromo-phenyl)-(8-methyl-quinazolin-4-yl)-amine,
Compound 57: (3,4-Dimethoxy-phenyl)-(8-methyl-quinazolin-4-yl)-amine,
Compound 58: (3-Chloro-6-hydroxy-phenyl)-(8-methyl-quinazolin-4-yl)-amine,
Compound 59: (2-Methoxy-4-methyl-phenyl)-(8-methyl-quinazolin4-yl)-amine,
Compound 60: (4-Chloro-phenyl)-(8-methyl-quinazolin-4-yl)-amine,
Compound 61: (8-Methyl-quinazolin-4-yl)-phenyl-amine
Compound 62: (3-Chloro-phenyl)-(8-methyl-quinazolin-4-yl)-amine,
Compound 63: (4-Fluoro-phenyl)-(8-methyl-quinazolin-4-yl)-amine,
Compound 64: (3,5-Bis-fluoromethyl-phenyl(8-methyl-quinazolin-4-yl)-amine,
Compound 65: (3-Bromo-phenyl)-(8-methyl-quinazolin-4-yl)-amine,
Compound 66: (3,5-Bis-fluoromethyl-phenyl)-(6-methyl-quinazolin-4-yl)-amine,
Compound 67: (4-Chloro-phenyl)-(6-methyl-quinazolin-4-yl)-amine,
Compound 68: Phenyl-[4-(8-methyl-quinazolin-4-yl-amino)-phenyl]-methanone,
Compound 69: (3-Bromo-phenyl)-(7-fluoro-quinazolin-4-yl)-amine,
Compound 70: (3-Chloro-phenyl)-(7-fluoro-quinazolin-4-yl)-amine,
Compound 71: (3-Ethyl-phenyl)-quinazolin-4-yl-amine,
Compound 72: (4-lodo-phenyl)-quinazolin-4-yl-amine,
Compound 73: (4-Phenoxy-phenyl)-quinazolin-4-yl-amine,
Compound 74: (4-Fluoro-phenyl)-(7-fluoro-quinazolin-4-yl)-amine,
Compound 75: (3-Fluoro-phenyl)-quinazolin-4-yl-amine,
Compound 76: (2,4-Dichloro-phenyl)-quinazolin-4-yl-amine,
Compound 77: (3-Hydroxy-phenyl)-quinazolin-4-yl-amine,
Compound 78: (3-Fluoro-4-methyl-phenyl)-(6-methyl-quinazolin-4-yl)amine,
Compound 79: (3-Fluoro-phenyl)-(6-methyl-quinazolin-4-yl)-amine,
Compound 80: (3-Chloro-6-methyl-phenyl)-(7-fluoro-quinazolin-4-yl)-amine,
Compound 81: Biphenyl-4-yl-quinazolin-4-yl-amine,
Compound 82: (3,4-Dimethoxy-phenyl)-quinazolin-4-yl-amine,
Compound 83: (6-Fluoro-quinazolin-4-yl)-(2-methyl-phenyl)-amine,
Compound 84: Phenyl-quinazolin-4-yl-amine,
Compound 85: (3-Chloro-6-methyl-phenyl)-quinazolin-4-yl-amine,
Compound 86: (4-Ethyl-phenyl)-quinazolin-4-yl)-amine,
Compound 87: (3-Chloro-phenyl)-quinazolin-4-yl-amine,
Compound 88: (4-Fluoro-phenyl)-quinazolin-4-yl-amine,
Compound 89: (3-Ethyl-phenyl)-(6-methyl-quinazolin-4-yl)-amine,
Compound 90: (7-Fluoro-quinazolin-4-yl phenyl-amine,
Compound 91: (3-Chloro-2-methyl-phenyl(6-methyl-quinazolin-4-yl)-amine,
Compound 92: (4-Butyl-phenyl)-(6-methyl-quinazolin-4-yl)-amine,
Compound 93: (4-Hydroxy-6-methyl-phenyl)-quinazolin-4-yl-amine,
Compound 94: (3-Nitro-6-methyl-phenyl)-quinazolin-4-yl-amine,
Compound 95: (3-Chloro-2-methyl-phenyl)quinazolin-4-yl-amine,
Compound 96: (4-Ethyl-phenyl)-(6-methyl-quinazolin-4-yl)-amine,
Compound 97: (4-Chloro-6-methyl-phenyl)-quinazolin-4-yl-amine,
Compound 98: (4-Bromo-phenyl)-quinazolin-4-yl-amine,
Compound 99: (3-Chloro-6-methyl-phenyl)-(8-methyl-quinazolin-4-yl)-amine,
Compound 100: (3-Chloro-6-methyl-phenyl)-(6-methyl-quinazolin-4-yl)-amine,
Compound 101: (3,4-Dimethoxy-phenyl)-(6-methyl-quinazolin-4-yl)-amine,
Compound 102: (3,4-Dimethoxy-phenyl)-(6,7-dimethoxy-quinazolin-4-yl)-amine,
Compound 103: (6-Methyl-quinazolin-4-yl)-(4-trifluoromethyl-phenyl)-amine,
Compound 104: (2-Fluoro-phenyl)-(7-fluoro-quinazolin-4-yl)-amine,
Compound 105: (3-Chloro-phenyl)-(6,7-dimethoxy-2-methyl-quinazolin-4-yl)-amine,
Compound 106: (6,7-Dimethoxy-quinazolin-4-yl)-(3-trifluoromethyl-phenyl)-amine,
Compound 107: (3-Bromo-phenyl)-(6-nitro-quinazolin-4-yl)-amine,
Compound 108: Biphenyl-4-yl-(8-methyl-quinazolin-4-yl)-amine,
Compound 109: (8-Methyl-quinazolin-4-yl)-(4-trifluoromethyl-phenyl)-amine,
Compound 110: (2,4-Dichloro-phenyl)-(6-methyl-quinazolin-4-yl)-amine,
Compound 111: (4-Trifluoromethyl-phenyl)-quinazolin-4-yl-amine,
Compound 112: (2,3-Dimethyl-phenyl)-(6-methyl-quinazolin-4-yl)-amine,
Compound 113: (3,5-Bistrifluoromethyl-phenyl)-(7-fluoro-quinazolin-4-yl)-amine,)
Compound 114: (2,4-Dimethyl-phenyl)-(6-methyl-quinazolin-4-yl)-amine,
Compound 115: Benzo[1,3]-dioxol-5-yl-quinazolin-4-yl-amine,
Compound 116: (6,7-Dimethoxy-quinazolin-4-yl)-(4-nitro-phenyl)-amine,
Compound 117: (6-Methyl-quinazolin-4-yl)-(2,4,5-Trichloro-phenyl)-amine,
Compound 118: (3-Chloro-5-fluoro-phenyl(6,7-dimethoxy-quinazolin-4-yl)-amine,
Compound 119: (3-Bromo-phenyl)-(6-nitro-7-fluoro-quinazolin-4-yl)-amine,
Compound 120: N⁴-(3-Bromo-phenyl)-6-nitro-quinazoline-4,7-diamine,
Compound 121: N-[4-(3-Bromo-phenylamino)-7-cyclobutane-carbonylamino-quinazolin-6-yl]-cyclobutanecarboxylic acid amide,
Compound 122: N-[4-(3-Bromo-phenylamino)7-cyclopentane-carbonylamino-quinazolin-6-yl]-cyclopentanecarboxylic acid amide,
Compound 123: N-[7-Acetylamino-5-(3-bromo-phenylamino)quinazolin-6-yl]-acetamide,
Compound 124: 3-(6,7-dimethoxy-quinazolin-4-yl-amino-benzonitrile,
Compound 125: (3-Chloro-4-fluoro-phenyl)-[7-methoxy-6-(3-morpholin-4-yl-propoxy)-quinazolin-4-yl]-amine.
   and/or pharmaceutically acceptable salts of these compounds.

Within said methods the quinazoline compounds of the general formula (I) and/or pharmaceutically acceptable salts thereof are administered in a dosage corresponding to an effective concentration in the range of 0.01-50 μM, preferably in the range of 0.02-10 μM, more preferably in the range of 0.03-1 μM, and most preferably in the range of 0.04-0.1 μM.

Pharmaceutical Compositions

In a further aspect the present invention relates to pharmaceutical compositions comprising at least one compound of the general formula (I) as an active ingredient together with a pharmaceutically acceptable carrier, excipient and/or diluents.

The quinazoline compounds of the present invention are basic and form pharmaceutically acceptable salts with organic and inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

It is also possible to obtain acid addition salts with amino acids like methionine, tryptophane, lysine or arginine, especially with quinazoline compounds of the general formula (I) substituted by a carboxylic acid residue.

Depending upon the substituents of the inventive quinazoline compounds, one may be able to form salts with bases, too. Thus, for example, if there are carboxylic acid substituents in the molecule, salts may be formed with inorganic as well as organic bases such as, for example, NaOH, KOH, NH₄OH, tetraalkylammonium hydroxide, and the like.

The compounds of the general formula (I) can also be administered in form of their pharmaceutically active salts optionally using substantially nontoxic pharmaceutically acceptable carriers, excipients or diluents. The medications of the present invention are prepared in a conventional solid or liquid carrier or diluents and a conventional pharmaceutically-made adjuvant at suitable dosage level in a known way. The preferred preparations are in administratable form which is suitable for oral application. These administratable forms, for example, include pills, tablets, film tablets, coated tablets, capsules, powders and deposits.

Furthermore, the subject of the present invention also includes pharmaceutical preparations for parenteral, including dermal, intradermal, intragastrical, intracutaneous, intravasal, intravenous, intramuscular, intraperitoneal, intranasal, intravaginal, intrabuccal, percutaneous, rectal, subcutaneous, sublingual, topical or transdermal application, which in addition to typical vehicles and diluents contain a quinazoline compound of the general formula (I) and/or a pharmaceutically acceptable salt thereof as active ingredient.

Within the disclosed methods the pharmaceutical compositions of the present invention, containing quinazoline derivatives of the general formula (I) as active ingredients, will typically be administered in a mixture with suitable carrier materials selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent in the inventive composition.

Suitable binders include starch, gelatin, natural sugars, corn sweeteners, natural and synthetic gums such as acacia, sodium alginate, carboxymethylcellulose, polyethylene glycol and waxes. Among the lubricants, there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. antihistaminic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidifies.

Also included are solid form preparations which are intended to be converted, shortly before use, to liquid form preparations for either oral or parenteral administration. Such liquid forms include solutions, suspensions and emulsions.

The inventive quinazoline compounds of the present invention may also be deliverable transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refers to the active ingredients dispersed or solubilized in a hydrophillic semi-solid matrix.

Powders for constitution refers to powder blends containing the active ingredients and suitable diluents which can be suspended in water or juices.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluents in the composition can range from about 5 to about 95% by weight of the total composition, preferably from about 25 to about 75%, more preferably from about 30 to about 60% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 2 to about 20% by weight of the composition, more preferably from about 5 to about 10% by weight.

Binders characterize substances that bind or “glue” powders together and make them cohesive by forming granules, thus serving as the “adhesive” in the formulation. Binders add cohesive strength already available in the diluent or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant refers to a substance added to the dosage form to enable the tablet; granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and D,L-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents are materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

### DESCRIPTION OF FIGURES

FIG. 1A+1B: show the inhibition of HCMV replication by using several quinazoline derivatives at different concentrations.

FIG. 2: shows the inhibition of HCMV replication in an AD 169 plaque reduction assay.

FIG. 3: Replication of UL97-deleted HCMV in the presence of pUL97 inhibitors. Plaque assays were performed with HCMV mutant AD169deIUL97, carrying a deletion in the UL97 gene, in the presence of 0.1% DMSO (no inhib.), 50 nM NGIC-I, 10 μM compound 37 or 2 μM CDV as indicated. Replication of AD169deIUL97 was efficiently blocked by the polymerase inhibitor CDV, but not by the pUL97 inhibitors NGIC-I and compound 37.

### EXPERIMENTAL PART

UL97 Kinase-Assay on Immobilon Plate

The effect of quinazoline derivatives on the activity of the viral kinase UL-97 was tested. This kinase is derived from human cytomegalovirus (HCMV) (Marschall, M., et al., J. Gen Virol. 2001, 82,1439-1450.). The UL-97 gene was cloned into a baculovirus vector in order to produce GST (glutathione S-transferase) fusion protein. Insect cells (Sf9) were infected and GST-UL-97 purified via glutathione affinity columns according to standard procedures.

UL-97 Kinase Reaction

The UL-97 kinase reaction was performed as described (Marschall M. et aL, J. Gen. Virol. 2001, 82, 1439-1450). Briefly, 10 μl Assay buffer (3 μM ATP, 60 μg/ml myelin basic protein (MBP) as substrate), 1,0 μCi gamma[³³P]ATP and 10 μp, Basic buffer (20mM Tris-HCl 7.5, 500 μM MnCl₂, 1 mM DTT (dithiothreitol)) were given to the test tube before adding various concentrations of the quinazoline derivatives. The reaction was started by adding 0.2 μl UL97 kinase, purified from infected Sf9-insect cells as described above. The total volume was adjusted with Basic buffer to a final volume of 30 μl. The reaction mix was incubated for 1 hr at 30° C. For negative control, 10 μl 0.1 M EDTA (ethylene diamine tetraacetate) was added to reaction mix before addition of UL-97 protein kinase. The reaction was stopped by addition of 10 μl 0.1 M EDTA.

Measuring Incorporation of Radioactivity

An Immobilon plate (Millipore) was rinsed with 50 μl methanol/well. Following addition of 100 μl 0.1 M EDTA, 20 μl of each kinase reaction mix was added to one well of the Immobilon plate. Each well was washed 4× with 250 μl 0.75% phosphoric acid and 1× with 50 μl methanol. After addition of 50 μl scintillation cocktail (Roth, Germany) per well incorporation of radioactivity was measured using a Betareader (Wallac) and enzymatic activity calculated.

HCMV Infection:

To examine the effect of the quinazoline derivatives on HCMV replication, HCMV infections were performed in a cellular system.

Human foreskin fibroblasts (HFFs) were grown to subconfluency in 12 well plates and infected with 0.25 tissue culture infectious doses of HCMV AD-169 carrying a reporter gene (HCMV based antiviral assay). Quinazoline derivatives (stocks in DMSO (Dimethylsulfoxide)) were diluted in culture medium to the concentrations indicated and added to the cells immediately after virus adsorption. The success of infection (reporter gene expressing cells) and the lack of cytotoxicity of the compound (confluent cell layer) was monitored by microscopy. After seven days, cell layers were harvested, lysed and subjected to the automated fluorometry measurement of reporter gene activity. Each panel refers to a determination in quadruplicate (infection in duplicate, lysate preparation and measurement in duplicate).

Toxicity Assay:

Toxicity of the quinazoline derivatives was measured by incubating HEK 293 cells with 10 μM of each derivative. The corresponding amount of DMSO served as control.

Plaque Reduction Assay:

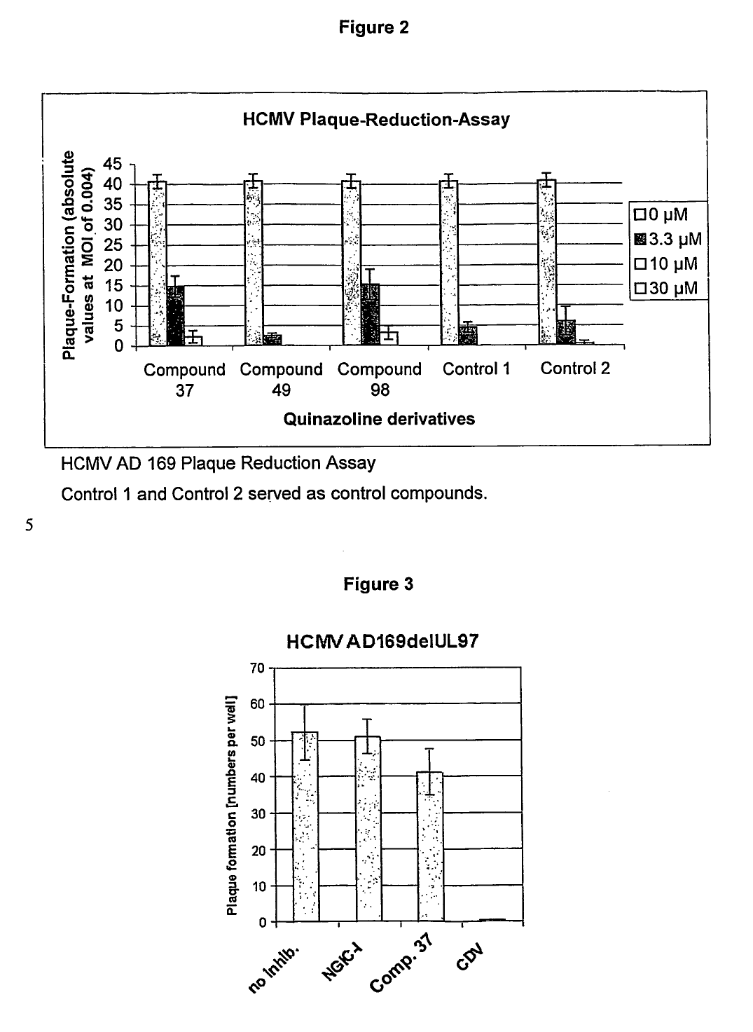

HCMV reference strain AD 169 was used at an MOI (multiplicity of infection) of 0.004 for the infection of HFFs in 12 well plates as described in FIG. 2. After virus adsorption, the infected cell layers were overlayed with fresh medium containing the compound concentration as indicated in FIG. 2 and 0.3% agarose. After eight days postinfection, a staining of virus plaques was performed by the use of 1% crystal violet. Quantification was achieved by microscopic counting in quadruplicate (infection in duplicate, counting in duplicate).

Furthermore, clinical isolates of HCMV like R5, R3 and R2 were used for the infection of HFFs in a plaque reduction assay under the conditions described above. Parallel experiments on phenotype characterization , gene sequencing and UL 97-in-cell-activity assay (GCV phosphorylation) with cloned UL 97 were performed (Marschall, M., et al., J. Gen Virol. 2001, 82, 1439-1450).

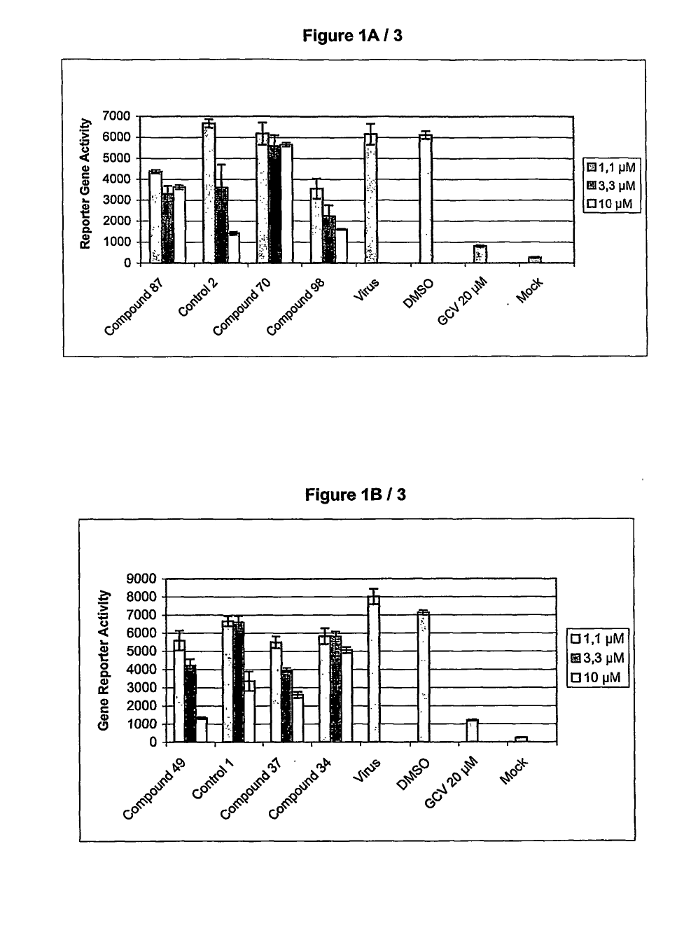

Results (See Also FIGS. 1A., 1B and 2):

The data summarized in table 1 show, that representatives of the class of quinazoline derivatives like (3-Bromo-phenyl)-quinazolin-4-yl-amine (compound 2), (4-Bromo-phenyl)-(6,7-dimethoxy-quinazolin-4-yl)-amine (compound 31), (3-Chloro-5-hydroxy-phenyl)-(6-methyl-quinazolin-4-yl)-amine (compound 34), (3,4-Dichloro-phenyl)-quinazolin-4-yl-amine (compound 37), (4-Bromo-phenyl)-(6-methyl-quinazolin-4-yl)-amine (compound 43), (3,4-Dichloro-phenyl)-(6-methyl-quinazolin-4-yl)-amine (compound 49), (3-Chloro-phenyl)-(7-fluoro-quinazolin-4-yl)-amine (compound 70), (4-lodo-phenyl)-quinazolin-4-yl-amine (compound 72), (3-Chloro-phenyl)-quinazolin-4-yl-amine (compound 87), (4-Fluoro-phenyl)-quinazolin-4-yl-amine, (compound 88), (3-Ethyl-phenyl)-(6-methyl-quinazolin-4-yl)-amine (compound 89) and (4-Bromo-phenyl)-quinazolin-4-yl-amine (compound 98) are potent inhibitors of UL 97-kinase activity (see Table 1). Furthermore, it was shown, that some quinazoline derivatives inhibit efficiently UL 97 in intact cells.

TABLE 1
Summary of the results of selected compounds according to the present invention:

| | | HCMV AD 169 | | | UL 97 | |
|---|---|---|---|---|---|---|
| | | Replication [%] | HCMV AD 169 | UL 97 | GCV [%] growth | Viability [%] |
| Compound | Structure | of control at 3 μM | IC 50 [μM] | IC 50 [μM] | at conc. [μM] | at 10 μM |
| Compound 2 | 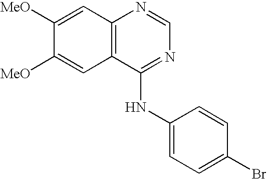 | 70 | n.d. | 0.40 | 47% 10 μM | 98 |
| Compound 31 | 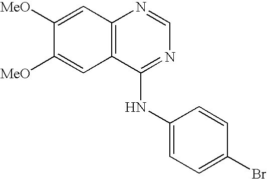 | 60 | n.d. | 1.20 | n.d. | n.d. |
| Compound 34 | 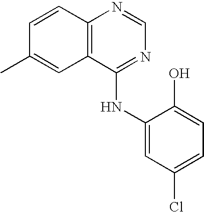 | n.d. | n.d. | 0.30 | n.d. | n.d. |
| Compound 37 | 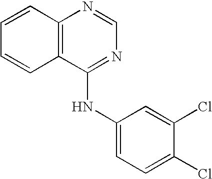 | 45 | 3.0 | 0.04 | 50% at 20 μM 80% at 10 μM | 77 |
| Compound 43 | 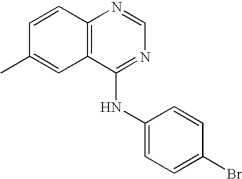 | 20 | n.d. | >0.10 | n.d. | n.d. |
| Compound 49 | 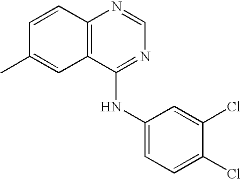 | 20 | 2.5 | 0.02 | 80% at 10 μM | 78% |
| Compound 70 | 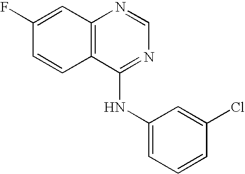 | n.d. | n.d. | 0.10 | n.d. | n.d. |
| Compound 72 | 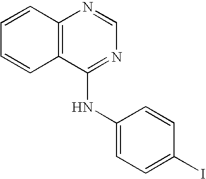 | n.d. | n.d. | 0.20 | n.d. | n.d. |
| Compound 84 | 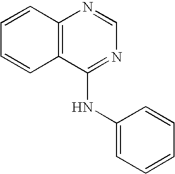 | n.d. | n.d. | 0.30 | n.d. | n.d. |
| Compound 87 | 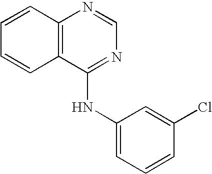 | n.d. | n.d.. | 0.70 | n.d. | n.d. |
| Compound 88 | 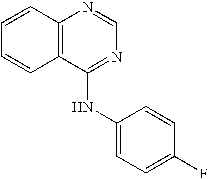 | n.d. | n.d. | 0.20 | n.d. | n.d. |
| Compound 98 | 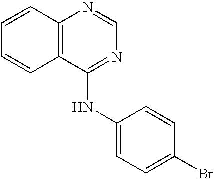 | 30 | 2.0 | 0.17 | 60% at 20 μM 80% at 10 μM | 75 |

Table 2 shows results obtained with selected quinazoline derivatives like the half maximal inhibition constant values (IC₅₀)-values on UL 97 activity. Furthermore, (IC₅₀)-values of HCMV replication was measured for some compounds in a cellular system. Some quinazoline derivatives protect HEK 293 cells ectopially expressing UL 97 against toxic concentrations of GCV (10 and 20 μM respectively).

Furthermore, compounds belonging to the class of quinazoline derivatives have been identified as potent inhibitor of HCMV replication in cell culture in two different assay systems (see FIG. 1A, 1B and 2).

The tested quinazoline derivatives (3,4-Dichloro-phenyl)-quinazolin-4-yl-amine (compound 37), (3,4-Dichloro-phenyl)-(6-methyl-quinazolin-4-yl)-amine (compound 49) and (4-Bromo-phenyl)-quinazolin-4-yl-amine (compound 98) show IC₅₀-values between 2 μM and 3 μM in the replication of the HCMV strain AD 169 in HFF cells and thus are more potent as the gold standard ganciclovir (IC₅₀=3-4 μM) (see FIG. 2). Furthermore, the IC₅₀-values for these compounds is between 2.5 and 5 μM in the plaque reduction assay. These IC₅₀-values are better than the IC₅₀-value of the gold standard ganciclovir, which is between 4-6 μM.

For the quinazoline derivatives 3,4-Dichloro-phenyl)-quinazolin-4-yl-amine (compound 37) and (3,4-Dichloro-phenyl)-(6-methyl-quinazolin-4-yl)-amine (compound 49) no signs of toxicity were observed up to 30 μM.

Test on Clinical Isolates:

Three clinical HCMV strains were investigated on replication in the presence of the selected quinazoline derivatives 3,4-Dichloro-phenyl)-quinazolin-4-yl-amine (compound 37), (3,4-Dichloro-phenyl)-(6-methyl-quinazolin-4-yl)-amine (compound 49) and (4-Bromo-phenyl)-quinazolin-4-yl-amine (compound 98). The clinical strain R2 is GCV (Ganciclovir) resistant and CDV (Cidofovir)-resistant clinical HCMV strain with IC 50 values of 34 μM (GCV) and 4.2 μM (CDV) respectively,. The clincical isolate R3, which is partially GCV resistant and CDV sensitive shows IC 50 values of 20 μM (GCV) and 0.35 μM (CDV). Furthermore, the clinical isolate R5, which is GCV sensitive and CDV sensitive with IC 50 values of 4.1 μM (GCV) and 0.7 μM (CDV), was used.

Replication of all three isolates (measured by plaque formation assays) and of Ad169 was efficiently blocked to the same degree by quinazolines derivatives. Additionally, a quinazoline-concentration-dependent inhibition of the above-mentioned three clinicical isolates (R2, R3 and R5) could be observed by not showing any toxicity up to 10 μM.

The quinazoline compounds of the present invention are UL 97-inhibitors and effectively block UL 97 at sub- and low-micromolar concentrations. Of all the other kinases, such as AbI, EGFR, InsR, Jnk, Akt, Cdk1, MAPK, p70S6K, ckit, Lck, PDGF, Met, Src, PCK and RIP tested in a selectivity panel only the EGFR kinase was inhibited by some of the quinazoline derivatives. For the other tested kinases no relevant inhibition of the kinase activity in the presence of 10 μM compound compared to the DMSO control was observed (data not shown).

Furthermore, the quinazoline compounds are low or non-toxic up to concentrations of 10 μM in HEK 293 cells and HFF cells.

Inhibition of HCMV Replication Depends on pUL97.

To confirm the conclusion that pUL97 is the target for the antiviral activity of quinazolines, we performed an experiment with an UL97-deleted mutant of HCMV (AD169deIUL97) (Prichard, M. N., Gao, N., Jairath, S., Mulamba, G., Krosky, P., Coen, D. M., Parker, B. O., and Pari, G. S., J. Virol. 1990, 73, 5663). As measured by plaque reduction assay, AD169deIUL97 virus possessed a drastic replication deficiency to about 1-5% compared to the parental virus but could eventually be grown to low-titre stocks and used for infection assays (Marschall, M., Stein-Gerlach, M., Freitag, M., Kupfer, R., van den Bogaard M., Stamminger, T., J. Gen Virol. 2002, 83,1013-1023). In plaque reduction assays, AD169deIUL97 virus was tested for sensitivity against quinazoline compound 37 [(3,4-Dichloro-phenyl)-quinazolin-4-yl-amine], indolocarbazole NGIC-I and CDV (inhibitor of viral DNA polymerase).
Compound 37 and NGIC-I had only little or no effect on viral replication, while CDV treatment resulted in complete inhibition (FIG. 3). This result further underlines that pUL97 is the main target of quinazoline antiviral activity.

1. A compound having the general formula (I): 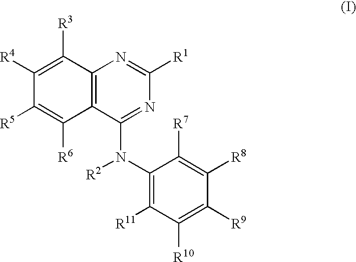
wherein:
R¹ is selected from the group consisting of:
—H and C₁-C₆-alkyl, aryl, or 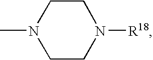
wherein R¹⁸ is selected from the group consisting of 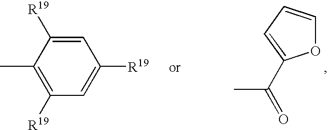
wherein R¹⁹ is independently selected from the group consisting of —H, —F, —Cl, —Br, —I, —NO₂ or —CF₃,
R² is selected from the group consisting of:
—H and C₁-C₆-alkyl,
R³, R⁴, R⁵ and R⁶ are independently selected from the group consisting of:
—H, C₁-C₆-alkyl, C₁-C₆-alkoxy, phenoxy, —F, —Cl, —Br, —I, —OH, —CN, —NR¹²R^{12′}, —N═N—R¹³, —NH—C(O)—R¹⁴, —NO₂, —C≡C—R¹⁵, —C(R²⁰)₃, or —CH(R²⁰)₂ or 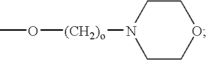
wherein o is selected to an integer from 0 to 6; and wherein
R¹² and R^{12′}are independently selected from the group consisting of:
—H and C₁-C₆-alkyl,
R¹³ and R¹⁴ are independently selected from the group consisting of
—H, C₁-C₆-alkyl,
—(CH₂)ₙ—R¹⁶, wherein n is selected to be an integer from 1 to 6 and R¹⁶ is selected from the group consisting of:
—OH, —NH₂, or —CN,
—(CH₂)ₘ—CH═CH₂, wherein m is selected to be an integer from 0 to 6, 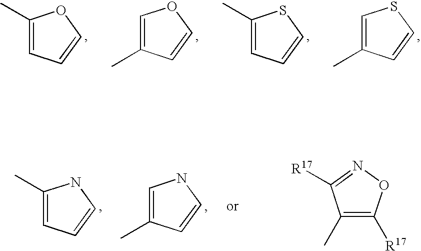
wherein R¹⁷ is selected from the group consisting of
—H, C₁-C₆-alkyl,
C₃-C₆-cycloalkyl,
phenyl substituted cyclopropyl, wherein the phenyl group is optionally substituted by one or two substituents R¹⁸, and R¹⁸ is independently selected from the group consisting of:
—F, —Cl, —Br, —I, —CN, —OH, —NH₂,
R¹⁵ is selected from the group consisting of:
—H and C₁-C₆-alkyl,
or wherein R⁴ and R⁵ together form one of the ring systems represented by the formulas 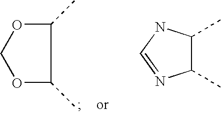
and
R²⁰ is independently selected from —F, —Cl, and —Br,
R⁷, R⁸, R⁹, R¹⁰, and R¹¹ are independently selected from the group consisting of:
H, C₁-C₆-alkyl, C₁-C₆-alkoxy, —F, —Cl, —Br, —I, —OH, —CN, —NR¹²R^{12′}, —N═N—R¹³, —NH—C(O)—R¹⁴, —NO₂, —C≡C—R¹⁵, —C(R²⁰)₃, or —CH(R²⁰)₂ or 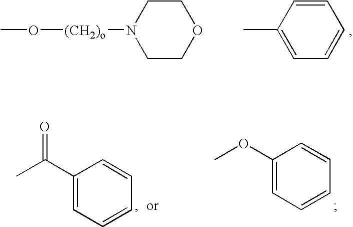
wherein o is selected to an integer from 0 to 6, and
wherein R¹², R^{12′}, R¹³, R¹⁴, R¹⁵, and R²⁰ represent groups as defined for R³ to R⁶, and
or wherein
R⁸ and R⁹ together form a together form a ring system represented by the formulas 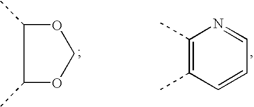
and/or pharmaceutically acceptable salts thereof.

2. Compound according to claim 1, wherein
R¹ is selected from the group consisting of —H or C₁-C₄-alkyl, and preferably is —H or methyl.

3. Compound according to claim 1, wherein
R² is selected from the group consisting of —H or C₁-C₄-alkyl, and preferably is —H.

4. Compound according to claim 1, wherein
R³, R⁴, R⁵ and R⁶ are independently selected from the group consisting of:
—H, C₁-C₆-alkyl, C₁-C₆-alkoxy, phenoxy, —F, —Cl, —Br, —I, —OH, —CN, —NR¹²R^{12′}—NH—C(O)—R¹⁴ —NO₂, —CF₃ or 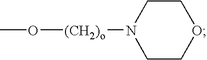
wherein o is selected to an integer from 0 to 4, preferably
from 2 to 4, most preferably is 3, and wherein
R¹² and R^{12′} are independently selected from the group consisting of —H or methyl,
R¹⁴ is selected from the group consisting of C₁-C₆-alkyl,
—(CH₂)ₘ—CH═CH₂, wherein m is selected to be an integer from 0 to 2 and preferably is 0, cyclopropyl, cyclobutyl, cyclopentyl, and cyclohexyl, and phenyl substituted cyclopropyl,
or wherein R⁴ and R⁵ together form a ring system represented by the formula

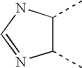

5. Compound according to claim 1, wherein
R⁷, R⁸, R⁹, R¹⁰, and R¹¹ are independently selected from the group consisting of:
—H. C₁-C₆-alkyl, C₁-C₆-alkoxy, —F, —Cl, —Br, —I, —OH, —N═N—R¹³, —NH—C(O)—R¹⁴, —NO₂, —C≡C—H, 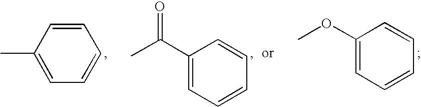
wherein
R¹³ and R¹⁴ are independently selected from the group consisting of
C₁-C₆-alkyl, and preferably are methyl or ethyl,
—(CH₂)ₙ—R¹⁶, wherein n is selected to be an integer from 1 to 6 and R¹⁶ is selected from the group consisting of:
—NH₂ and —CN,
—CH═CH₂, 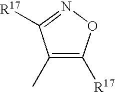
wherein R¹⁷ is selected from the group consisting of —H and methyl,
cyclopropyl, cyclobutyl, cyclopentyl, cyclohexyl, phenyl substituted cyclopropyl,
or wherein R⁸ and R⁹ together form one of the ring systems represented by the formulas

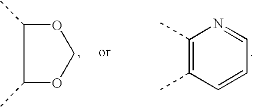

6. Compound according to claim 1, wherein
R³ and R⁶ represent —H and
R⁴ and R⁵ are independently selected from the group consisting of:
—H, C₁-C₄-alkyl, and preferably are —H or methyl.

7. Compound according to claim 1, wherein
R⁷, R⁸, R⁹, R¹⁰, and R¹¹ are independently selected from the group consisting of:
—H, C₁-C₆-alkyl, C₁-C₆-alkoxy, —F, —Cl, —Br, —I, —OH, —CN, and
—N¹²R^{12′},
wherein R¹² and R^{12′}are independently selected from —H or methyl and preferably are both —H.

8. Compound according to claim 1, wherein
R⁷, R⁸, R⁹, R¹⁰, and R¹¹ are independently selected from the group consisting of:
—H, —F, —Cl, —Br, and —I.

9. Compound according to claim 1, wherein
at least two of the groups R⁷, R⁸, R⁹, R¹⁰, and R¹¹ are —H, preferably three or two of the groups R⁷, R⁸, R⁹, R¹⁰, and R¹¹ are —H.

10. Compound according to claim 1, wherein four of the groups R⁷, R⁸, R⁹, R¹⁰, and R¹¹ are —H.

11. Compound according to claim 1, wherein R⁷, R⁹, R¹⁰, and R¹¹ are —H, or wherein R⁷, R¹⁰ and R¹¹ are —H, or wherein R⁸ and R¹⁰ are —H.

12. Compound according to claim 1, wherein those groups out of the group R⁷, R⁸, R⁹, R¹⁰, and R¹¹ which are not —H are selected from the group consisting of —F, —Cl, —Br and —I.

13. Compound according to claim 1, wherein the compound is selected from the group consisting of:
(Compound 1:) (3-Nitro-phenyl)-quinazolin-4-yl-amine,
(Compound 2:) (3-Bromo-phenyl)-quinazolin-4-yl-amine,
(Compound 3:) (6,7-Dimethoxy-quinazolin-4-yl)-[(3-(3,5-dimethyl-isoxyzol-4-ylazo)-phenyl]-amine,
(Compound 4:) Furan-2-carboxylic acid [4-(6,7-dimethoxy-quinazolin-4-yl-amino)-phenyl]-amide,
(Compound 5:) Furan-2-carboxylic acid [3-(6,7-dimethoxy-quinazolin-4-yl-amino)-phenyl]-amide,
(Compound 6:) 2-Cyano-N-[4-(6,7-dimethoxy-quinazolin-4-yl-amino)-phenyl]acetamide,
(Compound 7:) 2-Cyano-N-[3-(6,7-dimethoxy-quinazolin-4-yl-amino)-phenyl]acetamide,
(Compound 8:) (3-Bromo-phenyl)-(6-methoxy-quinazolin-4-yl)-amine,
(Compound 9:) (3-Bromo-phenyl)-(6-methyl-quinazolin-4-yl)-amine,
(Compound 10:) 6-Amino-hexanoic acid [4-(6,7-dimethoxy-quinazolin-4-yl)-phenyl]-amide,
(Compound 11:) 6-Amino-hexanoic acid [3-(6,7-dimethoxy-quinazolin-4-yl)-phenyl]-amide,
(Compound 12:) (3-Bromo-phenyl)-(6,7-dimethoxy-quinazolin-4-yl)-amine,
(Compound 13:) (6,7-Dimethoxy-2-methyl-quinazolin-4-yl)-(3-nitro-phenyl)-amine,
(Compound 14:) (6,7-Dimethoxy-2-methyl-quinazolin-4-yl)-(4-nitro-phenyl)-amine,
(Compound 15:) N-[4-(3-Bromo-phenylamino)-quinazolin-6-yl]-propionamide,
(Compound 16:) (3-Bromo-phenyl)-(6,7-diethoxy-quinazolin-4-yl)-amine,
(Compound 17:) (6,7-diethoxy-quinazolin-4-yl)-(3-hydroxy-phenyl)-amine,
(Compound 18:) N-[4-(3-Bromo-phenylamino)-quinazolin-6-yl]-propargylamide,
(Compound 19:) (3-Choro-phenyl)-(6,7-dimethoxy-quinazolin-4-yl)-amine,
(Compound 20:) N⁴-(3-Bromo-phenyl)-N⁶-methyl-quinazoline-4,6,7-triamine,
(Compound 21:) (3-Bromo-4-methoxy-phenyl)-(6,7-dimethoxy-quinazolin-4-yl)-amine,
(Compound 22:) N⁴-(3-Bromo-phenyl)-quinazoline-4,6,7-triamine,
(Compound 23:) (3,5-Bis-trifluormethyl-phenyl-quinazolin-4-yl)-amine,
(Compound 24:) (4-Fluoro-phenyl)-(6-methyl-quinazolin-4-yl)-amine,
(Compound 25:) (3-Chloro-phenyl)-(6-methyl-quinazolin-4-yl)-amine,
(Compound 26:) (6-Methyl-quinazolin-4-yl)-phenyl-amine,
(Compound 27:) (3,4-Dimethyl-phenyl)-(6-methyl-quinazolin-4-yl)-amine,
(Compound 28:) (2-Hydroxy-4-methyl-phenyl)-(6-methyl-quinazolin-4-yl)-amine,
(Compound 29:) (6,7-Dimethoxy-quinazolin-4-yl)- (4-fluoro-phenyl)-amine,
(Compound 30:) (3,4-Dimethyl-phenyl)-(6,7-dimethoxy-quinazolin-4-yl)-amine,
(Compound 31:) (4-Bromo-phenyl)-(6,7-dimethoxy-quinazolin-4-yl)-amine,
(Compound 32:) Benzo[1,3]-dioxol-5-yl-(6,7-dimethoxy.quinazolin-4-yl)-amine,
(Compound 33:) (3-Bromo-phenyl)-(6,7-dimethoxy-2-methyl-quinazolin-4-yl)-amine,
(Compound 34:) (3-Chloro-5-hydroxy-phenyl)-(6-methyl-quinazolin-4-yl)-amine,
(Compound 35:) N-[4-(Quinazolin-4-yl-amino)-phenyl]-acetamide,
(Compound 36:) Phenyl-[4-(quinazolin-4-yl-amino)-phenyl]-methanone,
(Compound 37:) (3,4-Dichloro-phenyl)-quinazolin-4-yl-amine,
(Compound 38:) (4-Chloro-2-hydroxy-phenyl)-quinazolin-4-yl-amine,
(Compound 39:) (2-Hydroxy-4-methyl-phenyl)-quinazolin-4-yl-amine,
(Compound 40:) (3,4-Dimethyl-phenyl)-quinazolin-4-yl-amine,
(Compound 41:) Benzo[1,3]-dioxol-5-yl-(6-methyl-quinazolin-4-yl)-amine,
(Compound 42:) (3-Chloro-4-methyl-phenyl)-(6-methyl-quinazolin-4-yl)-amine,
(Compound 43:) (4-Bromo-phenyl)-(6-methyl-quinazolin-4-yl)-amine,
(Compound 44:) N-[4-(6-Methyl-quinazolin-4-yl-amino)-phenyl]-acetamide,
(Compound 45:) (4-lodo-phenyl)-(6-methyl-quinazolin-4-yl)-amine,
(Compound 46:) Phenyl-[4-(6-methyl-quinazolin-4-yl-amino)-phenyl]-methanone,
(Compound 47:) (4-Phenoxy-phenyl)-quinazolin-4-yl-amine,
(Compound 48:) (3-Hydroxy-phenyl)-(6-methyl-quinazolin-4-yl)-amine,
(Compound 49:) (3,4-Dichloro-phenyl)-(6-methyl-quinazolin-4-yl)-amine,
(Compound 50:) N-[4-(8-Methyl-quinazolin-4-yl-amino)-phenyl]-acetamide,
(Compound 51:) (4-lodo-phenyl)-(8-methyl-quinazolin-4-yl)-amine,
(Compound 52:) (4-Phenoxy-phenyl)-(8-methyl-quinazolin-4-yl)-amine,
(Compound 53:) (3,4-Dichloro-phenyl)-(8-methyl-quinazolin-4-yl)-amine,
(Compound 54:) Benzo[1,3]-dioxol-5-yl-(8-methyl-quinazolin-4-yl)-amine,
(Compound 55:) (3-Chloro-4-methyl-phenyl)-(8-methyl-quinazolin-4-yl)-amine,
(Compound 56:) (4-Bromo-phenyl)-(8-methyl-quinazolin-4-yl)-amine,
(Compound 57:) (3,4-Dimethoxy-phenyl)-(8-methyl-quinazolin-4-yl)-amine,
(Compound 58:) (3-Chloro-6-hydroxy-phenyl)-(8-methyl-quinazolin-4-yl)-amine,
(Compound 59:) (2-Methoxy-4-methyl-phenyl)-(8-methyl-quinazolin-4-yl)-amine,
(Compound 60:) (4-Chloro-phenyl)-(8-methyl-quinazolin-4-yl)-amine,
(Compound 61:) (8-Methyl-quinazolin-4-yl)-phenyl-amine
(Compound 62:) (3-Chloro-phenyl)-(8-methyl-quinazolin-4-yl)-amine,
(Compound 63:) (4-Fluoro-phenyl)-(8-methyl-quinazolin-4-yl)-amine,
(Compound 64:) (3,5-Bis-fluoromethyl-phenyl)-(8-methyl-quinazolin-4-yl)-amine,
(Compound 65:) (3-Bromo-phenyl)-(8-methyl-quinazolin-4-yl)-amine,
(Compound 66:) (3,5-Bis-fluoromethyl-phenyl)-(6-methyl-quinazolin-4-yl)-amine,
(Compound 67:) (4-Chloro-phenyl)-(6-methyl-quinazolin-4-yl)-amine,
(Compound 68:) Phenyl-[4-(8-methyl-quinazolin-4-yl-amino)-phenyl]-methanone,
(Compound 69:) (3-Bromo-phenyl)-(7-fluoro-quinazolin-4-yl)-amine,
(Compound 70:) (3-Chloro-phenyl)-(7-fluoro-quinazolin-4-yl)-amine,
(Compound 71:) (3-Ethyl-phenyl)-quinazolin-4-yl-amine,
(Compound 72:) (4-lodo-phenyl)-quinazolin-4-yl-amine,
(Compound 73:) (4-Phenoxy-phenyl)-quinazolin-4-yl-amine,
(Compound 74:) (4-Fluoro-phenyl)-(7-fluoro-quinazolin-4-yl)-amine,
(Compound 75:) (3-Fluoro-phenyl)-quinazolin-4-yl-amine,
(Compound 76:) (2,4-Dichloro-phenyl)-quinazolin-4-yl-amine,
(Compound 77:) (3-Hydroxy-phenyl)-quinazolin-4-yl-amine,
(Compound 78:) (3-Fluoro-4-methyl-phenyl)-(6-methyl-quinazolin-4-yl)-amine,
(Compound 79:) (3-Fluoro-phenyl)-(6-methyl-quinazolin-4-yl)-amine,
(Compound 80:) (3-Chloro-6-methyl-phenyl)-(7-fluoro-quinazolin-4-yl)-amine,
(Compound 81:) Biphenyl-4-yl-quinazolin-4-yl-amine,
(Compound 82:) (3,4-Dimethoxy-phenyl)-quinazolin-4-yl-amine,
(Compound 83:) (6-Fluoro-quinazolin-4-yl)-(2-methyl-phenyl)-amine,
(Compound 84:) Phenyl-quinazolin-4-yl-amine,
(Compound 85:) (3-Chloro-6-methyl-phenyl)-quinazolin-4-yl-amine,
(Compound 86:) (4-Ethyl-phenyl)-quinazolin-4-yl)-amine,
(Compound 87:) (3-Chloro-phenyl)-quinazolin-4-yl-amine,
(Compound 88:) (4-Fluoro-phenyl)-quinazolin-4-yl-amine,
(Compound 89:) (3-Ethyl-phenyl)-(6-methyl-quinazolin-4-yl)-amine,
(Compound 90:) (7-Fluoro-quinazolin-4-yl)-phenyl-amine,
(Compound 91:) (3-Chloro-2-methyl-phenyl)-(6-methyl-quinazolin-4-yl)-amine,
(Compound 92:) (4-Butyl-phenyl)-(6-methyl-quinazolin-4-yl)-amine,
(Compound 93:) (4-Hydroxy-6-methyl-phenyl)-quinazolin-4-yl-amine,
(Compound 94:) (3-Nitro-6-methyl-phenyl)-quinazolin-4-yl-amine,
(Compound 95:) (3-Chloro-2-methyl-phenyl)-quinazolin-4-yl-amine,
(Compound 96:) (4-Ethyl-phenyl)-(6-methyl-quinazolin-4-yl)-amine,
(Compound 97:) (4-Chloro-6-methyl-phenyl)-quinazolin-4-yl-amine,
(Compound 98:) (4-Bromo-phenyl)-quinazolin-4-yl-amine,
(Compound 99:) (3-Chloro-6-methyl-phenyl)-(8-methyl-quinazolin-4-yl)-amine,
(Compound 100:) (3-Chloro-6-methyl-phenyl)-(6-methyl-quinazolin-4-yl)-amine,
(Compound 101:) (3,4-Dimethoxy-phenyl)-(6-methyl-quinazolin-4-yl)-amine,
(Compound 102:) (3,4-Dimethoxy-phenyl)-(6,7-dimethoxy-quinazolin-4-yl)-amine,
(Compound 103:) (6-Methyl-quinazolin-4-yl)-(4-trifluoromethyl-phenyl)-amine,
(Compound 104:) (2-Fluoro-phenyl)-(7-fluoro-qu inazolin-4-yl)-amine,
(Compound 105:) (3-Chloro-phenyl)-(6,7-dimethoxy-2-methyl-quinazolin-4-yl)-amine,
(Compound 106:) (6,7-Dimethoxy-quinazolin-4-yl)-(3-trifluoromethyl-phenyl)-amine,
(Compound 107:) (3-Bromo-phenyl)-(6-nitro-quinazolin-4-yl)-amine,
(Compound 108:) Biphenyl-4-yl-(8-methyl-quinazolin-4-yl)-amine,
(Compound 109:) (8-Methyl-quinazolin-4-yl)-(4-trifluoromethyl-phenyl)-amine,
(Compound 110:) (2,4-Dichloro-phenyl)-(6-methyl-quinazolin-4-yl)-amine,
(Compound 111:) (4-Trifluoromethyl-phenyl)-quinazolin-4-yl-amine,
(Compound 112:) (2,3-Dimethyl-phenyl)-(6-methyl-quinazolin-4-yl)-amine,
(Compound 113:) (3,5-Bistrifluoromethyl-phenyl)-(7-fluoro-quinazolin-4-yl)-amine,)
(Compound 114:) (2,4-Dimethyl-phenyl)-(6-methyl-quinazolin-4-yl)-amine,
(Compound 115:) Benzo[1,3]-dioxol-5-yl-quinazolin-4-yl-amine,
(Compound 116:) (6,7-Dimethoxy-quinazolin-4-yl)-(4-nitro-phenyl)-amine,
(Compound 117:) (6-Methyl-quinazolin-4-yl)-(2,4,5-Trichloro-phenyl)-amine,
(Compound 118:) (3-Chloro-5-fluoro-phenyl)-(6,7-dimethoxy-quinazolin-4-yl)-amine,
(Compound 119): (3-Bromo-phenyl)-(6-nitro-7-fluoro-quinazolin-4-yl)-amine,
(Compound 120:) N⁴-(3-Bromo-phenyl)-6-nitro-quinazoline-4,7-diamine,
(Compound 121:) N-[4-(3-Bromo-phenylamino)-7-cyclobutane-carbonylamino-quinazolin-6-yl]-cyclobutanecarboxylic acid amide,
(Compound 122:) N-[4-(3-Bromo-phenylamino)-7-cyclopentane-carbonylamino-quinazolin-6-yl]-cyclopentanecarboxylic acid amide,
(Compound 123:) N-[7-Acetylamino-5-(3-bromo-phenylamino)quinazolin-6-yl]-acetamide,
(Compound 124:) 3-(6,7-dimethoxy-quinazolin-4-yl-amino-benzonitrile,
(Compound 125:) (3-Chloro-4-fluoro-phenyl)-[7-methoxy-6-(3-morpholin-4-yl-propoxy)-quinazolin-4-yl]-amine.
and/or pharmaceutically acceptable salts thereof.

14. Compound according to claim 1, for use as a pharmaceutically active agent.

15. Use of at least one compound according to claim 1 as a pharmaceutically active agent.

16. Use according to claim 15 for the prophylaxis and/or treatment of infectious diseases, including opportunistic diseases.

17. Use according to claim 16, wherein the infectious disease is caused by herpes viruses.

18. Use according to claim 17, wherein the herpes virus is selected from the group comprising: herpes simplex viruses, varicello viruses, cytomegalo viruses, muromegalo viruses, roseolo viruses, lymphocrypto viruses and rhadino viruses.

19. Use according to claim 18, wherein the herpes virus is a cytomegalo virus.

20. Use according to claim 19, wherein the herpes virus is human cytomegalo virus (HCMV).

21. Use of at least compound according to claim 1, for the preparation of a pharmaceutical composition for the prophylaxis and/or treatment of infectious diseases, including opportunistic diseases.

22. Use according to claim 21, wherein said infectious disease is caused by herpes viruses.

23. Use according to claim 22, wherein the herpes virus is selected from the group comprising: herpes simplex viruses, varicello viruses, cytomegalo viruses, muromegalo viruses, roseolo viruses, lymphocrypto viruses and rhadino viruses.

24. Use according to claim 23, wherein the herpes virus is a cytomegalo virus.

25. Use according to claim 24, wherein the herpes virus is human cytomegalo virus (HCMV).

26. Use according to claim 15, wherein said infectious disease is herpes.

27. Use according to claim 15, wherein the herpes virus is a drug resistant virus strain.

28. Use of at least one compound according to claim 1, as an inhibitor for a protein kinase.

29. Use according to claim 28, wherein the protein kinase is a herpesviral kinase.

30. Use according to claim 29, wherein the herpesviral kinase is UL 97.

31. Use of at least one compound according to claim 1, is administered in a dosage corresponding to an effective concentration in the range of 0.01-50 mm.

32. Use of at least one compound according to claim 1, is administered in combination with further therapeutic compounds.

33. Use according to claim 32, wherein the further therapeutic compounds are selected from the group consisting of:
Ganciclovir, foscarnet, cidofovir, valganciclovir, ganciclovir implants, fomivirsen, penciclovir and valaciclovir.

34. Pharmaceutical composition comprising at least one compound according to claim 1, as an active ingredient, together with at least one pharmaceutically acceptable carrier, excipient and/or diluent.

35. Pharmaceutical composition according to claim 34 further comprising an additional therapeutic agent selected from the group consisting of:
Ganciclovir, foscarnet, cidofovir, valganciclovir, ganciclovir implants, fomivirsen, penciclovir and valaciclovir.

36. Method for preventing and/or treating infectious diseases, including opportunistic diseases in a mammal, especially in a human, which method comprises administering to the mammal an amount of at least one compound as defined in claim 1, effective to prevent and/or treat said infectious disease and/or opportunistic infection.

37. Method according to claim 36, wherein said infectious disease is caused by a herpes virus.

38. Method for preventing and/or treating herpesviral infections and/or associated diseases in a mammal, including a human, by administering a pharmaceutically effective amount of at least one compound as defined in claim 1, to said mammal, wherein said compound inhibits at least partially the activity of a herpesviral kinase, preferably of UL 97.

39. Method according to claim 36, wherein the herpes virus is selected from the group comprising herpes simplex viruses, varicello viruses, cytomegalo viruses, muromegalo viruses, roseolo viruses, lymphocrypto viruses and rhadino viruses.

40. Method according to claim 36, wherein the herpes virus is a cytomegalo virus.

41. Method according to claim 40, wherein the herpes virus is human cytomegalo virus.

42. Method according to claim 39, wherein the herpes virus is a drug resistant virus strain.

43. Method according to claim 36, wherein said infectious disease or herpesviral infection is herpes.

44. Method for preventing and/or treating infectious diseases, including opportunistic diseases in a mammal, especially in a human, which method comprises administering to the mammal an amount of at least one compound as defined in claim 1 is administered in a dosage corresponding to an effective concentration in the range of 0.01-50 mm to prevent and/or treat said infectious disease and/or opportunistic infection.

45. Method for preventing and/or treating infectious diseases, including opportunistic diseases in a mammal, especially in a human, which method comprises administering to the mammal an amount of at least one compound as defined in claim 1 is administered in combination with at least one further therapeutic compound, selected from the group consisting of Ganciclovir, foscarnet, cidofovir, valganciclovir, ganciclovir implants, fomivirsen, penciclovir and valaciclovir to prevent and/or treat said infectious disease and/or opportunistic infection.
